(19) United States
(12) Patent Application Publication
(10) Pub. No.: US 2022/0331584 A1
(43) Pub. Date: Oct. 20, 2022

(54) SACRAL LEAD FOR STIMULATION AND/OR SENSING SIGNALS WITHIN A PATIENT

(71) Applicant: Medtronic, Inc., Minneapolis, MN (US)

(72) Inventors: Sarah J. Offutt, Golden Valley, MN (US); Katie C. Bittner, White Bear Lake, MN (US); Julia P. Slopsema, Minneapolis, MN (US); Leonid M. Litvak, Los Angeles, CA (US); Lisa M. Jungbauer Nikolas, Lino Lakes, MN (US); Katelynn M. Johnson, New Brighton, MN (US); Lance Zirpel, Lino Lakes, MN (US); Simeng Zhang, Golden Valley, MN (US)

(21) Appl. No.: 17/658,978

(22) Filed: Apr. 12, 2022

Related U.S. Application Data
(60) Provisional application No. 63/175,426, filed on Apr. 15, 2021.

(51) Int. Cl. A61N 1/05; A61N 1/36; A61B 5/00; A61B 5/06

(52) U.S. Cl. CPC A61N 1/0551; A61N 1/36007; A61B 5/6851; A61B 5/6877; A61B 5/064; A61N 1/36135

(57) ABSTRACT

A sacral lead system including a sacral lead configured to for insertion within a sacral foramen of a patient. The sacral lead supports one or more electrodes which may be configured as one or more stimulation electrodes and/or one or more sensing electrodes. The sacral lead is configured to deliver a stimulation signal to a patient using at least one stimulation electrode and sense an evoked signal produced in response to the stimulation signal using at least one sensing electrode. The sacral lead system may be configured to position the at least one stimulation electrode and/or the at least one sensing electrode within, dorsal, or ventral to the sacral foramen. The sacral lead system may include stimulation circuitry configured to generate the stimulation signal and sensing circuitry configured to receive a signal indicative of the evoked signal.

This application claims the benefit of U.S. Provisional Application Ser. No. 63/175,426 (filed Apr. 15, 2021), which is entitled “SACRAL LEAD FOR STIMULATION AND/OR SENSING SIGNALS WITHIN A PATIENT” and is incorporated by reference herein in its entirety.

### TECHNICAL FIELD

The disclosure relates to a medical lead system and use for delivery of therapy to a patient.

### BACKGROUND

Urinary and fecal incontinence, and urinary retention, (e.g., an inability to control bladder and bowel functions) are problems that afflict people of all ages, genders, and races. Various muscles, nerves, organs, and conduits within the pelvis cooperate to collect, store and release bladder and bowel contents. A variety of disorders may compromise urinary tract and bowel performance, and contribute to incontinence. Many of the disorders may be associated with aging, injury, or illness.

Urinary incontinence, such as, urgency incontinence, may originate from disorders of portions of the peripheral or central nervous system which control the bladder micturition reflex. Nerve disorders may also lead to overactive bladder activities and/or may prevent proper triggering and operation of the bladder. Furthermore, urinary incontinence may also result from improper communication between the nervous system and the urethra.

### SUMMARY

The disclosure describes a sacral lead system configured to deliver a stimulation signal (e.g., a stimulation waveform) to a sacral nerve of a patient. The sacral lead system includes a sacral lead configured to insert into a sacral foramen and supporting one or more electrodes. The sacral lead is configured to deliver a stimulation signal to a patient using the one or more electrodes as one or more stimulation electrodes, and sense an evoked signal using the one or more electrodes as one or more sensing electrodes. The evoked signal is produced by the patient in response to the stimulation signal. In examples, sacral lead system is configured to use at least one electrode of the one or more electrodes as both a stimulation electrode and a sensing electrode. In examples, the sacral lead is configured to position at least one of the one or more electrodes within or ventral to the sacral foramen. The sacral lead may be configured to position the at least one electrode dorsal to the sacral foramen (e.g., dorsal to an anterior opening of the sacral foramen) and intracorporeal to the patient. The sacral lead system may include stimulation circuitry configured to generate the stimulation signal and sensing circuitry configured to receive a signal indicative of the evoked signal.

In examples, a method of sensing and stimulation with a sacral lead comprises: delivering a stimulation signal through one or more stimulation electrodes using one or more electrodes, wherein the one or more electrodes are configured to operate as the one or more stimulation electrodes; and sensing, following delivery of the stimulation signal, an evoked signal with one or more sensing electrodes using the one or more electrodes, wherein the one or more electrodes are configured to operate as the one or more sensing electrodes, wherein at least one of the stimulation electrodes is located within, dorsal, or ventral to a foramen of a sacrum of a patient, and wherein at least one of the sensing electrodes is located within, dorsal, or ventral to the foramen of the sacrum of the patient, wherein a lead body of the sacral lead supports at least some portion of the one or more electrodes, wherein the lead body is configured to position the at least some portion of the one or more electrodes within or ventral to the foramen.

In examples, a method of sensing and stimulation with a sacral lead comprises: extending, using a lead body of the sacral lead, the sacral lead through a sheath lumen of an introducer sheath configured to extend dorsal to or within the foramen, wherein the introducer sheath defines one or more windows defining one or more openings in a sheath wall of the introducer sheath, wherein the lead body supports at least some portion of the one or more electrodes, and wherein the lead body is configured to position the at least some portion of the one or more electrodes within or ventral to the foramen; aligning, using the lead body, at least one window of the introducer sheath with at least one of the one or more electrodes; delivering a stimulation signal through one or more stimulation electrodes using the one or more electrodes when the one or more electrodes are configured to operate as the one or more stimulation electrodes; and sensing, following delivery of the stimulation signal, an evoked signal with one or more sensing electrodes using the one or more electrodes when the one or more electrodes are configured to operate as the one or more sensing electrodes, wherein the evoked signal includes a signal generated by a signal source of the patient in response to delivery of the stimulation signal, wherein the signal source includes at least one of a muscle of the patient or a nerve of the patient, wherein at least one of the stimulation electrodes is located within, dorsal, or ventral to a foramen of a sacrum of a patient, and wherein at least one of the sensing electrodes is located within, dorsal, or ventral to the foramen of the sacrum of the patient.

In examples, a sacral lead system comprises: one or more electrodes, wherein the one or more electrodes are configured to operate as one or more stimulation electrodes and one or more sensing electrodes; a sacral lead including a lead body, wherein the lead body supports at least some portion of the one or more electrodes, and wherein the lead body is configured to position at least some portion of the one or more electrodes within, dorsal to, or ventral to a foramen of a sacrum of a patient; and processing circuitry configured to: deliver, using the one or more electrodes configured to operate as the one or more stimulation electrodes, a stimulation signal; and sense, following delivery of the stimulation signal, and using the one or more electrodes configured to operate as the one or more sensing electrodes, an evoked signal.

The details of one or more examples are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example system that manages delivery of neurostimulation to a patient to manage bladder and bowel dysfunction, such as overactive bladder, urgency, or urinary incontinence, retention.

FIG. 2 is a block diagram illustrating example configurations of a medical device which may be utilized in the system of FIG. 1.

FIG. 3 is a schematic diagram illustrating an example sacral lead and a sheath.

FIG. 4 is a schematic diagram illustrating the sacral lead of FIG. 3 positioned within a sacral foramen of a patient.

FIG. 5 is a schematic diagram illustrating the sacral lead of FIG. 3 and FIG. 4 in another position within the sacral foramen of the patient.

FIG. 6 is a schematic diagram illustrating the sacral lead of FIGS. 3-5 in an additional position within the sacral foramen of the patient.

FIG. 7 is a flowchart illustrating an example technique for using the sacral lead of FIGS. 3-6.

FIG. 8 is a schematic diagram illustrating an example arrangement of electrodes on the sacral lead of FIGS. 3-6.

FIG. 9 is a schematic diagram illustrating another example arrangement of electrodes on the sacral lead of FIGS. 3-6 and FIG. 8.

FIG. 10 is a schematic diagram illustrating the sheath of FIG. 3 including a first plurality of windows.

FIG. 11 is a schematic diagram illustrating the sheath of FIG. 3 including a second plurality of windows.

FIG. 12 is a schematic diagram of the sacral lead FIGS. 3-6 and FIGS. 8-11 including a fixation structure.

FIG. 13 is a schematic diagram of the sacral lead FIGS. 3-6 and FIGS. 8-12 including a helical element.

FIG. 14 is a schematic diagram illustrating the sheath of FIG. 3 including a plurality of windows with an angular offset.

FIG. 15 is a schematic diagram illustrating the sacral lead of FIGS. 3-6 and FIGS. 8-12 including a plurality of electrodes with an angular offset.

### DETAILED DESCRIPTION

The present disclosure is directed to devices, systems, and techniques for managing dysfunction of a patient and/or other patient conditions via electrical stimulation of the sacral nerve are described in this disclosure. The electrical stimulation therapy may include delivery of electrical stimulation to one or more sacral nerve sites via a medical device. Such electrical stimulation may be used to modify pelvic function to treat various patient conditions (e.g., urinary incontinence and fecal incontinence, and urinary retention) by modulating bladder and/or bowel functions. For ease, the examples are described with respect to bladder dysfunction (e.g., for urinary incontinence), but the techniques may be applicable to bowel dysfunction or fecal incontinence.

One type of therapy for treating bladder dysfunction, pain relief, and/or other therapeutic benefits includes delivery of electrical stimulation to a target site within a patient to cause a therapeutic effect during delivery of the electrical stimulation. The delivery of electrical stimulation may be continuous, may cycle on and off, or be on during certain times and off during certain times. This therapeutic effect may be sustained even for periods of time when the electrical stimulation is off. For example, delivery of electrical stimulation from an implantable medical device (IMD) to a target therapy site, e.g., a tissue site directly or indirectly involved with modulating the activity of a spinal nerve (e.g., a sacral nerve), a pudendal nerve, dorsal genital nerve, a tibial nerve, a saphenous nerve, an inferior rectal nerve, a perineal nerve, branches of any of the aforementioned nerves, roots of any of the aforementioned nerves, ganglia of any of the aforementioned nerves, or plexus of any of the aforementioned nerves, may provide a therapeutic effect for bladder dysfunction, such as a desired reduction in frequency of bladder contractions. In some cases, electrical stimulation (e.g., neuromodulation) of the sacral nerve may modulate afferent nerve activities to restore urinary function during the electrical stimulation.

Neuromodulation is generally conducted by delivering electrical stimulation to the nervous system of a patient by placing one or more stimulating electrodes substantially adjacent and/or proximate to targeted neural tissue. Sacral neuromodulation is a form of neuromodulation that generally provides peripheral nerve stimulation by delivering electrical stimulation to a sacral nerve, such as one of the spinal sacral nerves S1, S2, S3, and S4. Commonly, the sacral neuromodulation provides nerve stimulation to the spinal sacral nerve S3. The electrical stimulation may be delivered using a sacral lead positioned within or in the vicinity of a foramen of a sacrum (“sacral foramen”) of the patient. The sacral lead may be electrically connected to a medical device (e.g., a pulse generator) and configured to deliver the electrical stimulation via one or more stimulation electrodes supported by the sacral lead. The sacral lead may be inserted percutaneously into the patient under image guidance and anchored in place by fixation elements (e.g., tines and/or barbs) which contact soft tissue and/or bone (e.g., in the corresponding sacral foramen). The stimulation electrodes can be employed singly or in combination to produce an electrical field that interacts with the target nerve. Generally, closer proximities to the target nerve and the stimulation electrode(s) provides a higher likelihood for optimal effect. For examples, closer proximities may enable lower energy consumptions during delivery of the therapy, more programming options for delivery of the therapy, a reduced likelihood of potential side-effects, and other potentially improved clinical outcomes.

Because of this relationship between sacral lead placement and the efficacy of subsequent therapy, trial stimulations may be applied to the stimulation electrodes to evaluate the placement of the sacral lead. Patient responses to the trial stimulations may be observed to evaluate the placement. The position of the sacral lead within or in the vicinity of the sacral foramen may be adjusted to alter a position of the stimulation electrodes relative to the target nerve based on the evaluation.

The disclosure herein describes techniques and examples of sacral lead systems configured to stimulate a sacral nerve (e.g., via sacral neuromodulation) and sense an evoked signal generated in the patient in response to the stimulation. The evoked signal may be a neural response in adjacent nerves, muscle contractions within the pelvic floor, and distal contractions in the foot, and/or signals generated by the patient in response to the stimulation. For example, stimulation of sacral nerves through electrical leads implanted near sacral nerves via sacral neuromodulation may evoke a neural response in adjacent nerves, muscle contractions within the pelvic floor, and distal contractions in the foot. The neural response in nerves and activation/contraction of muscles evoked by electrical stimulation may be captured (e.g., or detected, sensed, measured, and the like) as an evoked signal that may be a composite signal comprising one or more distinct signals generated from one or more signal sources. For examples, a composite stimulation-evoked signal may comprise one or more stimulation-evoked signals generated by one or more signal sources in response to the electrical stimulation, e.g., one or more signals may come from one signal source or more than one signal source. A sensed composite stimulation-evoked signal may be a composite of signals from one or more nerves, one or more muscles, or at least one muscle and/or at least one nerve captured concurrently within a particular amount of time. In examples, a composite signal (e.g., composite stimulation-evoked signal) comprises two or more signals generated from one or more signal sources (e.g., in response to the electrical stimulation).

In some examples, a composite stimulation-evoked signal may be a composite of one or more stimulation-evoked signals from one or more sources, e.g., the one or more sources being one or more anatomical structures of the patient such as particular nerves and/or particular muscles. In other examples, a composite stimulation-evoked signal may be a composite of one or more stimulation-evoked signals from a single source, e.g., a single anatomical structure such as a particular nerve or particular muscle. For example, in response to stimulation, a nerve may generate both an evoked compound action potential (ECAP) signal and a reflex signal after a time delay. As another example, a muscle may activate and/or contract in response to stimulation, and over a period of time additional and/or a different subset of muscle fibers may contract and/or release, generating a first signal over a first period of time (corresponding to a first subset of muscle fibers activating/contracting) and a second signal over a second period of time (corresponding to a second subset of muscle fibers activating/contracting). In some examples, a composite stimulation-evoked signal may be a composite of one or more stimulation-evoked signals of the same type, or a different type, e.g., two ECAPs, an ECAP and an electromyogram (EMG), or the like.

In some examples, an evoked signal (e.g., a stimulation-evoked composite signal) may be a “direct stimulation-evoked signal” that is directly evoked, e.g., evoked by a signal source (e.g., nerve or muscle) in response to stimulation of that same signal source. In other examples, a stimulation-evoked signal (or stimulation-evoked composite signal) may be an “indirect stimulation-evoked signal” that is indirectly evoked, e.g., evoked by a signal source (e.g., nerve or muscle) in response to stimulation of a different part of the patient's anatomy. For example, a signal evoked by a distal contraction of the patient's foot, where the distal contraction is in response to stimulation of a sacral nerve, is an indirect stimulation-evoked signal. Throughout the disclosure, the term “evoked signal” encompasses any or all of direct, indirect, single, and/or composite stimulation-evoked signals unless further specified.

In some examples, the evoked signal may be a composite stimulation-evoked signal comprising a composite of signals generated by one or more signal sources in response to a stimulation signal (e.g., a stimulation waveform). The one or more signal sources may be one or more of one or more muscles of the patient, one or more nerves of the patient, or at least one muscle and at least one nerve of the patient.

A neural response in nerves and activation/contraction of muscles evoked by the electrical stimulation (e.g., a stimulation signal) may be captured (e.g., or detected, sensed, measured, and the like) as an evoked signal. An evoked signal may include one or more features that may indicate one or more aspects of electrical stimulation therapy delivery, such as a positioning of electrical lead(s) that provides effective therapy, e.g., electrical lead placement that improves symptoms and/or disease systems. Evoked signals, or a lack thereof, e.g., a lack of activation/response/contraction in response to electrical stimulation, may indicate a placement of the electrical lead that does not provide effective therapy, e.g., poor electrical lead placement and subsequent therapy. Additionally, captured evoked signals may be a composite of multiple signals evoked by multiple signal sources (e.g., nerves and/or muscles) in response to delivery of electrical stimulation therapy.

In accordance with one or more techniques of this disclosure, example electrical stimulation systems and example techniques may utilize evoked signals for determining one or more aspects of electrical stimulation therapy delivery, such as lead positioning, stimulation parameters, stimulation timing, and the like. For example, a medical device may output one or more electrical stimulation signals (e.g., waveforms) via one or more stimulation electrodes on a lead, and one or more sensing electrodes on the same lead or a different lead may sense one or more neural responses and/or one or more muscle activation/contraction responses as one or more stimulation-evoked signals. In some examples, the evoked signal may be a composite evoked signal that is a composite of signals generated by one or more signal sources, e.g., nerves and/or muscles, in response to the delivered electrical stimulation signals. For example, the evoked signal may be a composite of signals from one or more nerves, one or more muscles, one or more nerve fibers, or within, a nerve, or at least one muscle and at least one nerve (or nerve fiber) captured concurrently within a particular amount of time. The particular amount of time may be an amount of time starting when the electrical stimulation begins or ends, and ending after a predetermined amount of time has passed, or ending based on the composite stimulation-evoked signal, one or more of the constituent signals of the composite stimulation-evoked signal, or some other trigger such as a physiological response or patient-input response is received, or ending based on other criteria. In some examples, a composite evoked signal may be a composite of one or more signals generated by single signal source, e.g., at different times and captured within a particular amount of time. For example, delivery of an electrical stimulation signal may cause multiple responses from a single signal source, e.g., a muscle or nerve, and each response of the signal source may generate a signal (e.g., a stimulation-evoked signal).

In some examples, an evoked signal may comprise one or more EMG. In some examples, the evoked signal may comprise more than an EMG, e.g., a compound action potential such as an ECAP, a surface EMG, an MMG, a network excitability, and/or multiple signals of differing signal type evoked by one or more signal sources. In some examples, signal sources may include nerves such as sacral nerves, e.g., dorsal and ventral rami of sacral nerves, pudendal nerves, sciatic nerves, saphenous nerves, nerves in the sacral plexus, pelvic nerves, pelvic plexus nerves, pelvic splanchnic nerves, inferior hypogastic plexus nerves, lumbosacral trunk nerves, e.g., where the lumbosacral trunk joins sacral nerves, any sympathetic nerve fibers in the sympathetic chain of any of the above nerves or other nerves. In some examples, signal sources may include muscles such as an external anal sphincter muscle, coccygeus muscle, levator ani muscle group, bulbocavernosus and/or bulbospongiosus muscle, gluteal muscles, e.g., gluteal maximus, gluteal medius, and gluteal minimus, perineal muscles, ischiocavernosus muscles, puborectalis muscles, piriformis muscles, or any other muscles.

In some examples, the composite evoked signal may be a combination of any and/or all of the various signal sources. For example, an electrical stimulation signal may cause a nerve and/or muscle proximate to the stimulation signal to generate a response and other nerves or muscles, not necessarily proximate to the stimulation signal, may also generate responses. In some examples, an electrical stimulation signal may cause a proximate nerve to respond and/or directly activate one or muscles and causing those one or more muscles to respond. In some examples, the electrical stimulation signal may be applied to, or proximate to, the spinal cord, which may respond with a reflex and/or reflex signal, e.g., one or more nerve fibers may evoke one or more reflexes and/or reflex signals, which may be stimulation-evoked signals. In some examples, a reflex and/or reflex signal may be elicited and/or caused from a nerve proximate to the spinal cord, e.g., via stimulation applied to such proximate nerve or stimulation applied to, or proximate to, the spinal cord which then causes such proximate nerve to response to elicit the reflex and/or reflex signal. The composite stimulation-evoked signal may be a composite of signals from any of the multiple signal sources.

In some examples, a composite stimulation-evoked signal may include signal features indicative of the responses of one or more signal sources (e.g., nerves or muscles) that occur over a relatively long period of time, e.g., more than 5 milliseconds (ms), more than 10 ms, more than 20 ms, etc. In other words, a composite stimulation-evoked signal may contain information relating to the efficacy of electrical stimulation therapy from the responses of the signal source(s) and may occur over a relatively long period of time (e.g., a relatively long signal capture time window). For example, different signal sources may have different response times, e.g., neural responses versus muscle contractions, and the different sources may be located at different distances from both the electrical stimulation source (e.g., an electrode of a lead) and a sensor (e.g., which may be the same and/or a different electrode on the same and/or different lead, or a different sensor located within and/or external to the patient's body). In order to capture at least a portion or substantially all of each of the stimulation-evoked signals from the different signal sources, the signal capture time window may be longer than any single stimulation-evoked signal because of the varying response times, temporal signal lengths, and signal source distances. In some examples, the timing of the sensing/receipt of stimulation-evoked signals may depend on how fast a particular signal source activates, e.g., adjacent nerves may be the fastest (e.g., shortest response time) and a muscle or any a post-synaptic neural activation may be slower (e.g., have a longer response time). For example, a stimulation-evoked nerve response (e.g., neural signal) may be generated about 3 ms after stimulation, a muscle signal (e.g., generated by a muscle response such as a contraction, muscle activity, muscle electrical activity, or the like) may be generated about 15 ms after stimulation, and a reflex muscle response (e.g., contraction, activity, electrical activity, or the like) may be generated about 75 ms after stimulation. The timing of the sensing/receipt of stimulation-evoked signals may also depend on how close the signal source is to the sensing/capturing electrode, e.g., it takes some time for the signal to get to the electrode. In some examples, the composite stimulation-evoked signal may include a plurality of stimulation-evoked signals that “arrive” at a lead over a period of time and are received at least partially overlapping in time. In some examples, such component stimulation-evoked signals of the sensed/received composite stimulation-evoked signal may be distinguishable from each other via their respective signal features.

In some examples, a composite stimulation-evoked signal may provide more complete information regarding stimulation therapy efficacy, e.g., as opposed to capturing an individual signal and/or stimulation-evoked signal from one or more signal sources. For example, the ensemble of signal sources may respond differently than the sum of individual signal sources, e.g., there may be interactions between the source and/or sources generating the plurality of stimulation-evoked signals, and systems and/or techniques disclosed may include a signal capture time window that is long enough to capture the ensemble response as a composite stimulation-evoked signal.

In some examples, there may be a signal capture time delay because of the differing response time delays, and/or the signal capture delay may be a combination of the different distances and different response times of the one or more signal sources. In some examples, the composite stimulation-evoked signal that includes stimulation-evoked signals from the one or more signal sources may have a relatively long duration, e.g., at least 3 ms, at least 5 ms, at least 10 ms, at least 20 ms, etc. In some examples, the composite evoked signal may comprise signals of different types from different signal sources. For example, the composite evoked signal may comprise an ECAP signal generated relatively quickly after delivery of electrical stimulation signals, e.g., within 10 ms, and an EMG signal generated relatively slowly after delivery of electrical stimulation signals, e.g., after 5 ms, or after 3 ms, or after 1 ms. In some examples, the composite evoked signal may comprise signals from multiple signal sources that do not overlap in time. For example, the composite evoked signal may comprise an ECAP signal from a signal source relatively close to the sensor and/or electrode followed by an EMG signal or another ECAP signal from a different signal source that may be relatively far from the sensor and/or electrode, e.g., such that the ECAP from the close signal source is no longer present while the EMG signal and/or ECAP from the more distant signal source are received by the sensor and/or electrode. In some examples, the composite evoked signal may have an amplitude of one or more peaks that are greater than 1 millivolt (mV), or greater than 0.1 mV, or greater than 0.01 mV, or greater than 0.001.

In some examples, one or more signal sources may be located relatively far from an electrode (e.g., electrodes 19, 21, 30) and/or each other, e.g., at least 5 millimeters (mm) from the electrode and/or each other, at least 10 mm from the electrode and/or each other, at least 100 mm from the electrode and/or each other, at least 200 mm from the electrode and/or each other, at least 1 meter from the electrode and/or each other. For example, one or more signal sources may include a tibial nerve responding to sacral nerve stimulation.

The sacral lead system may be configured to deliver the stimulation signal using one or more stimulation electrodes supported by a sacral lead. The sacral lead may be configured to sense the evoked signal using one or more sensing electrodes supported by the sacral lead. In accordance with one or more techniques of this disclosure, the sacral lead may be utilized to deliver a stimulation signal and sense an evoked signal to determine one or more aspects of the electrical stimulation therapy delivery, such as sacral lead positioning, stimulation parameters, stimulation timing, and the like. The technique may include evaluating the evoked response to evaluate the current position of the sacral lead within the patient. In examples, the technique includes altering a position of the sacral lead within the patient based on the evoked response. It may be possible, in some examples, for one lead to include the stimulation electrodes and another lead to include the sensing electrodes.

The sacral lead supports one or more (e.g., a plurality) of electrodes on a lead body configured to extend within and/or through a sacral foramen of the patient. The one or more electrodes include at least one stimulation electrode configured to deliver a stimulation signal to a sacral nerve, and includes at least one sensing electrode configured to sense an evoked signal generated by the patient in response to the stimulation signal. In examples, the sensing electrode is proximal to the stimulation electrode on the lead body. In examples, an individual electrode within the plurality of electrodes may be configured to operate as the stimulation electrode and the sensing electrode. For example, the sacral lead may include a zeroth electrode as a most distal electrode in the plurality and include an nth electrode proximal to the zeroth electrode. The nth electrode may be configured to sense an evoked signal generated by a stimulation signal issued by the zeroth electrode in a first configuration of the sacral lead, and configured to deliver the stimulation signal (with a more distal electrode serving as the sensing electrode) in a second configuration of the sacral lead. The sacral lead may be configured to deliver the stimulation signal and/or sense the evoked signal using any combination of the one or more (e.g., the plurality) of electrodes. For example, the sacral lead may be configured to deliver a stimulation signal using the zeroth electrode and the nth electrode, and sense the evoked signal using another electrode in the plurality besides the zeroth electrode or the nth electrode.

As used herein, a “stimulation electrode” may refer to an electrode supported by a sacral lead and configured to emit a stimulation signal to tissues and/or a sacral nerve of a patient when the electrode is intracorporeal to the patient. The sacral lead may include a conductor configured to communicate the stimulation signal from stimulation circuitry of a medical device to the stimulation electrode. A “sensing electrode” may refer to an electrode supported by a sacral lead and configured to sense an evoked signal generated by the patient in response to a stimulation signal. The sacral lead may include a conductor configured to communicate a signal indicative of the evoked signal from the sensing electrode to sensing circuitry of a medical device. Hence, a given electrode of the sacral lead may be configured as stimulation electrode in some examples and configured as a sensing electrode in other examples. In some examples, the given electrode may be configured as both the stimulation electrode and the sensing electrode (e.g., when an evoked signal is chronologically subsequent to the stimulation signal). In some examples, a first electrode of the sacral lead may be configured to serve mainly as a sensing electrode and include structural distinctions from a second electrode configured to serve mainly as a stimulation electrode. For example, the first electrode may be configured to define a first input impedance greater than a second input impedance define by the second electrode. In some examples, the first electrode may define a first effective surface area less than a second effective surface area defined by the second electrode. As used here, an “effective surface area” may be a surface area which defines at least in part an input impedance of an electrode.

Within this disclosure, when the disclosure refers to a stimulation electrode, this may mean a single electrode configured as a stimulation electrode in some examples, and/or may mean a single electrode within one or more stimulation electrodes comprising the one or more electrodes of the sacral lead, wherein each of the one or stimulation electrodes is configured as a stimulation electrode. When the disclosure refers to a sensing electrode, this may mean a single electrode configured as a sensing electrode in some examples, and/or may mean a single electrode within one or more sensing electrodes comprising the one or more electrodes of the sacral lead, wherein each of the one or sensing electrodes is configured as a sensing electrode.

In examples, conductors of the sacral lead are configured to operably couple each electrode of the sacral lead to an external device. The external device may be configured to operably connect each electrode to either or both of the stimulation circuitry and/or the sensing circuitry, such that the external device may substantially dictate whether a given electrode operates as a stimulation electrode, a sensing electrode, or both a stimulation electrode and a sensing electrode. The external device may be operable by a clinician, such that the clinician may provide an input determining whether the given electrode operates as a stimulation electrode, a sensing electrode, or both a stimulation electrode and a sensing electrode. For example, the sacral lead may be configured such that the external device causes a first electrode to be used as a stimulation electrode and each of a second electrode and a third electrode to be used as a sensing electrode when the sacral lead is in a first position within a patient. The sacral lead may be configured such that the external device causes each of the first electrode and the second electrode to be used as a stimulation electrode and the third electrode to be used as a sensing electrode when the sacral lead is in a second position within a patient. Hence, the sacral lead may be configured to use each of any combination of electrodes as a stimulation electrode and each of any combination of electrodes as a sensing electrode, depending on the configuration of the external device. Thus, the sacral lead is configured such that a clinician may alter the stimulation and sensing functions of a given electrode based on a position of the sacral lead within a patient, or for other reasons.

The sacral lead is configured to position a stimulation electrode (e.g., at least one of the one or more electrodes configured as a stimulation electrode) within or ventral (e.g., distal) to a sacral foramen of the patient. In examples, the sacral lead is configured to position the stimulation electrode proximate the anterior opening of the sacral foramen. The sacral lead is configured to position the sensing electrode (e.g., at least one of the one or more electrodes configured as a sensing electrode) dorsal (e.g., proximal) to the anterior opening of the sacral foramen when the sacral lead positions the stimulation electrode proximate a sacral nerve of the patient. In examples, the sacral lead is configured to position the stimulation electrode in the vicinity of and/or substantially adjacent to a location where the sacral nerve exits the anterior opening of the sacral foramen. For example, the sacral lead may be configured to position the stimulation electrode slightly dorsal to, slightly ventral to, or substantially at the anterior opening of the sacral foramen to position the stimulation electrode proximate the sacral nerve.

In examples, the sacral lead is configured to position at least one stimulation electrode ventral to a posterior opening of the foramen and position at least one sensing electrode is located ventral to the posterior opening. The sacral lead may be configured to position at least one stimulation electrode ventral to an anterior opening of the foramen and position at least one sensing electrode ventral to a posterior opening of the foramen. The sacral lead may be configured to position at least one stimulation electrode within the foramen and position at least one sensing electrode ventral to an anterior opening of the foramen. The sacral lead may be configured to position at least one stimulation electrode ventral to a posterior opening of the foramen and position at least one sensing electrode dorsal to an anterior opening of the foramen. The sacral lead may be configured to position at least one stimulation electrode dorsal to an anterior opening of the foramen and position at least one sensing electrode ventral to a posterior opening of the foramen. In examples, the sacral lead is configured to position at least one stimulation electrode dorsal to an anterior opening of the foramen and position at least one sensing electrode dorsal to the anterior opening of the foramen.

The sacral lead is configured to position the sensing electrode intracorporeal to the patient when the stimulation electrode is positioned proximate the sacral nerve (e.g., when the stimulation electrode is positioned within or ventral to the sacral foramen of the patient). The sacral lead may be configured to position the sensing electrode within the sacral foramen when the stimulation electrode positions proximate the sacral nerve. In examples, the sacral lead is configured to position the sensing electrode dorsal to the sacral foramen when the stimulation electrode positions proximate the sacral nerve. In some examples, the sacral lead is configured to position the stimulation electrode ventral to the anterior opening of the sacral foramen and position the sensing electrode within and dorsal to the sacral foramen of the patient.

The disclosure includes example techniques for positioning the sacral lead within a patient using the stimulation electrode and the sensing electrode. The examples may include using programmable stimulation and sensing configurations that each comprises one or more electrodes. The examples may include using the sacral lead to position the stimulation electrode within or ventral to the sacral foramen of the patient and position the sensing electrode intracorporeal to the patient. Some examples use the sacral lead to position the stimulation electrode slightly dorsal to, slightly ventral to, or substantially at the anterior opening of the sacral foramen to position the stimulation electrode proximate a sacral nerve exiting the anterior opening. The example techniques may include using the sacral lead to deliver a stimulation signal via the positioned stimulation electrode and subsequently receive an evoked signal using the sensing electrode. The techniques may include using the sacral lead to alter a position of the stimulation electrode relative to the anterior opening of the sacral foramen in response to the evoked signal. The techniques may include using a sacral lead (e.g., a first sacral lead, such as a trial lead) to deliver one or more stimulation signals and/or receive one or more evoked signals as the sacral lead in inserted within the patient (e.g., inserted through the sacral foramen) to identify a target location within the patient (e.g., a target location for the one or more electrodes of the sacral lead. The techniques may include using a sacral lead (e.g., a second sacral lead, such as a lead configured to remain implanted in the patient) to deliver one or more stimulation signals and/or receive one or more evoked signals at the target location (e,g, when the one or more electrodes of the second sacral lead or the first sacral lead are substantially positioned at or in proximity to the target location).

As used herein, when a first object is described as dorsal to a second object, the first object is substantially between the second object and a dorsal surface of a patient. When a first object is described as ventral to a second object, the first object is substantially between the second object and a ventral surface of the patient. When a motion is described as ventral and/or ventrally, a motion is substantially toward the ventral surface of the patient. When a motion is described as dorsal and/or dorsally, a motion is substantially toward the dorsal surface of the patient.

In examples, the lead body of the sacral lead includes a distal portion comprising a distal end. The lead body may support (e.g., mechanically support) a plurality of electrodes, including a distal-most electrode positioned on the lead body between the distal end and every other electrode in the plurality. In examples, the techniques include using the sacral lead to position the distal-most electrode in a first position within or ventral to the sacral foramen of the patient and delivering a first stimulation signal via at least the distal-most electrode in the first position. The techniques may include using the sacral lead to receive a first evoked signal generated by the patient in response to the first stimulation signal delivered by the distal-most electrode in the first position. In examples, the techniques include using the sacral lead to displace the distal-most electrode to a second position ventral to the first position (e.g., ventral relative to the sacral foramen), and delivering a second stimulation signal using at least the distal-most electrode in the second position to generate a second evoked signal. The techniques may include using the sacral lead to position the distal-most electrode at the first position or the second position based on the first evoked signal and the second evoked signal.

The techniques may include using the sacral lead to position at least one of the one or more electrodes in the first position and delivering the first stimulation signal via the at least one electrode, and using the sacral lead to receive a first evoked signal generated by the patient in response to the first stimulation signal. The techniques may include using the sacral lead to displace the at least one electrode to a second position ventral to the first position and delivering a second stimulation signal using at least the one electrode. The techniques may include using the sacral lead to position the at least one electrode at the first position or the second position based on the first evoked signal and the second evoked signal.

Hence, the techniques may include establishing a position of the sacral lead based on evoked signals generated by the patient and received by the sacral lead in response to stimulation signals delivered to the patient by the lead. The evoked signals may be received by a sensing electrode supported by the lead at a position intracorporeal to the patient. The techniques may include using the sacral lead to displace at least one electrode (e.g., the distal-most electrode) at a third position ventral to the second position, receiving a third evoked signal, displacing the at least one electrode (e.g., the distal-most electrode) at a fourth position ventral to the third position, receiving a fourth evoked signal, and so on to provide a plurality of evoked signals, and using the sacral lead to position the at least one electrode (e.g., distal-most electrode) at one of the positions based on the plurality of evoked signals.

In examples, the sacral lead is configured to deliver a stimulation signal through the at least one electrode (e.g., the distal-most electrode) as well as through other electrodes proximal to the distal-most electrode. The sacral lead may be configured such that altering a position of the sacral lead relative to the anterior opening of the sacral foramen (e.g., altering a position of the at least one electrode (e.g., the distal-most electrode)) alters a proximity of the other electrodes relative to the sacral nerve. Thus, altering a position of the sacral lead relative to the anterior opening may increase the stimulation received by the sacral nerve from the one or more (e.g., plurality) of electrodes. This may, in turn, cause the evoked signal received in response to a stimulation signal to alter when the position of the sacral lead is altered. Hence, the sacral lead may be configured such that the evoked signal sensed by the intracorporeal sensing electrode serves as a proxy for the stimulation received by the sacral nerve from the one or more (e.g., plurality) of electrodes. Positioning the sacral lead to increase and/or optimize the stimulation received from the one or more (e.g., the plurality) of electrodes may increase the effectiveness of delivered therapy for the patient, reduce the operating power required by the sacral lead during operation, increase the likelihood of successful placement, and/or assist a clinician during the placement procedure.

In examples, the techniques include using the sacral lead of the sacral lead system to position one or more (e.g., a plurality) of the stimulation electrodes until the evoked signal achieves a threshold. The threshold may be based on the stimulation signals delivered by the stimulation electrodes, and/or based on a position of the sacral lead relative to a sacral nerve. For example, the sacral lead may be configured to be operably coupled to a processing circuitry (e.g., stimulation circuitry) of the sacral lead system configured to control a power of the stimulation signal issued via the stimulation electrodes. The threshold of the evoked signal may be based on the power of the stimulation signal. In examples, the sacral lead is configured to allow altering a position of the stimulation electrodes until the evoked signal threshold is achieved. In examples, the techniques include using the sacral lead to alter a position of the distal-most electrode ventrally relative to the anterior opening of the sacral foramen until an evoked signal generated by the patient in response to a stimulation signal achieves the evoked signal threshold. As used here, achieving the evoked signal threshold may refer to achieving a value within a certain range of a threshold. The value may be based on a discrete measured value of the evoked signal, an area associated with a waveform generated by the evoked signal, a shape parameter associated with a waveform generated by the evoked signal, or some other value having a magnitude based on a characteristic of the evoked signal.

In examples, the techniques include using the sacral lead to position the at least one electrode (e.g., the distal-most electrode) in a plurality of positions with each position successively more ventral to the anterior opening than a preceding position. The techniques may include continuing to position the at least one electrode in successively more ventral positions until the evoked signal achieves the threshold. In this way, the sacral lead may be configured to substantially indicate when a sufficient length of the sacral lead has been inserted within and/or through the sacral foramen of a patient, such that the clinician may limit the extent to which the sacral lead is ventrally displaced beyond the anterior opening of the sacral foramen. In some examples, a clinician uses the sacral lead to initially position the at least one electrode substantially adjacent the anterior opening (e.g., just dorsal to, substantially even with, or just ventral to the anterior opening) before using the sacral lead to position the at least one in successively more ventral positions. In some examples, the clinician uses the sacral lead to initially position the at least one electrode and a sensing electrode (e.g., one of the one or more electrodes which not the at least one electrode) dorsal to the anterior opening. In some examples, the clinician uses the sacral lead to initially position the at least one electrode ventral to the anterior opening and the sensing electrode ventral to the anterior opening. In some examples, the clinician uses the sacral lead to initially position the at least one electrode substantially at an edge of the anterior opening and the sensing electrode dorsal to the anterior opening.

The sacral lead system may be configured to position and/or displace the at least one electrode when the lead body of the sacral lead is positioned and/or displaced (e.g., by a clinician) within a patient e.g., when the at least one electrode is a specific electrode of the one or more electrodes). In examples, processing circuitry of the sacral lead system is configured to position and/or displace the at least one electrode by selecting (e.g., activating) or deselecting (e.g., deactivating) a given electrode in the one or more electrodes to act as the at least one electrode. For example, the processing circuitry may be configured to initially use (e.g., select and/or activate) a first electrode at a first location on the lead body such that the first electrode acts as the at least one electrode. The processing circuitry may be configured to subsequently use (e.g., select and/or activate) a second electrode at a second location on the lead body as the at least one electrode The processing circuitry may deselect and/or deactivate the first electrode when the processing circuitry uses the second electrode as the at least one electrode. Hence, in some examples, the at least one electrode may be positioned and/or displaced using the processing circuitry while the lead body and electrodes remain substantially stationary relative to the body of the patient.

The sacral lead is configured to be visible by an imaging modality (e.g., a fluoroscope) while within the patient. The sacral lead may be configured such that a location of one or more electrodes may be ascertained by a clinician when the one or more electrodes are within the patient. In examples, electrodes supported by the lead body of the sacral lead are configured for visibility using the imaging modality. In examples, the sacral lead includes one or more imaging markers configured to enhance visibility using the imaging modality, such that the location of the one or more electrodes may be ascertained by observing an image of the one or more markers. Thus, the sacral lead may be configured such that the clinician may use the imaging modality or evoked signals received to assess the location of at least one of the one or more electrodes (e.g., a distal-most electrode and/or other electrodes) of the sacral lead during an implantation procedure. In examples, at least one imaging marker is configured to be imaged by an imaging modality configured to image one or more anatomical features of a patient. The at least one imaging markers may be configured such that an image of the one or more anatomical features and the at least one imaging marker may be utilized (e.g., by a clinician, and/or the processing circuitry) to define a displacement between the at least one imaging marker and at least one of the one or more anatomical features of the patient.

In examples, and as described above, the evoked signal is a composite signal including a plurality of signals evoked by the stimulation signal. For example, the evoked signal may be a composite of signals from one or more nerves, one or more muscles, or at least one muscle and at least one nerve. In examples, the composite of signals includes two or more signals captured substantially concurrently within a particular amount of time. The particular amount of time may be an elapsed time period which commences based on a timing of the stimulation signal. For example, the particular amount of time may be an amount of time commencing when the stimulation signal begins or ends and/or an amount of time ending after a predetermined amount of time has passed. The particular amount of time may be based on the composite signal, one or more of the evoked signals, or some other trigger such as a physiological response, or other criteria.

In some examples, the composite signal (e.g., the evoked signal), may include signal features indicative of the response of one or more signal sources (e.g., nerves or muscles) that occur over a relatively long amount of time, e.g., more than 5 milliseconds (ms), more than 10 ms, more than 20 ms, etc. In some examples, the composite signal (e.g., the evoked signal), may include signal features indicative of the response of one or more signal sources that occur over a period of time less than or about 5 ms. In other words, an evoked signal may contain information relating to the efficacy of electrical stimulation therapy from the responses of a plurality of signal sources and may occur over a relatively long amount of time (e.g., a relatively long signal capture time window). For example, the multiple signal sources may have differing response times, e.g., neural responses versus muscle contractions, and the multiple sources may be located at different distances from an electrical stimulation source (e.g., a stimulation electrode) and an evoked signal sensor (e.g., the sensing electrode, and/or a different electrode on the same and/or different sacral lead, or a different sensor located within and/or external to the patient's body). In some examples, the composite signal may provide more complete information regarding stimulation therapy efficacy, e.g., as opposed to capturing an individual stimulation-evoked signal from each of multiple signal sources. For example, the ensemble of stimulation-evoked signal sources may respond differently than the sum of individual signal sources, and a system may include a signal capture time window that is long enough to capture the ensemble response as a composite stimulation-evoked signal.

In some examples, processing circuitry of the sacral lead system may be configured to determine features based on the captured composite signal, and therapy efficacy may be determined based on the features and/or a collection of features captured from a collection of patients. For example, machine learning may be used on a collection of features from patient composite signals and paired with stimulation outcome measures to build a classification algorithm that can predict patient therapy response outcome and therapy efficacy. In some examples, the predicted therapy efficacy may then be used to make therapy decisions, e.g., implant leads or not implant leads, choose an electrode configuration, tune stimulation parameters, and the like.

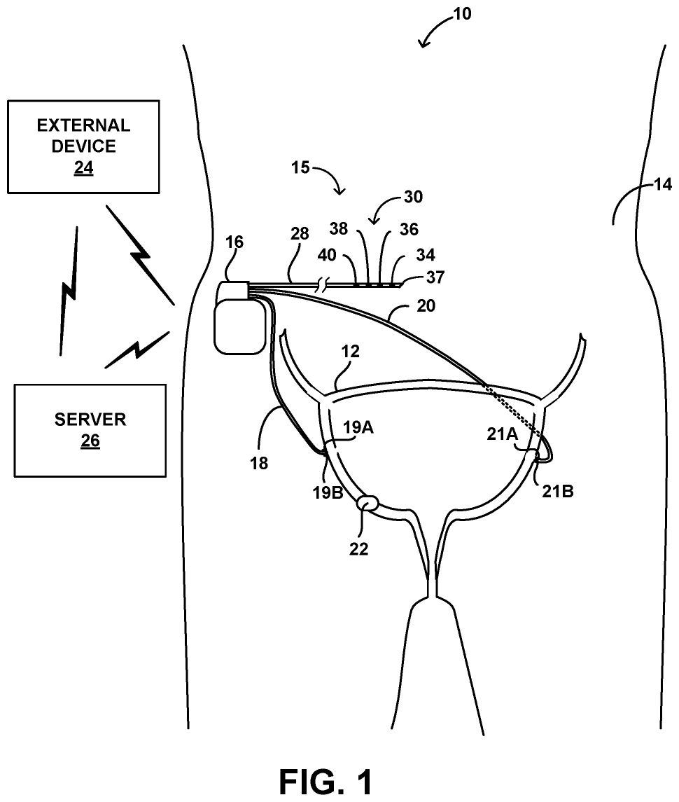

FIG. 1 is a conceptual diagram illustrating an example system 10 configured to manage delivery of neurostimulation to a patient 14 to manage bladder dysfunction, such as overactive bladder, urgency, or urinary incontinence. As shown in the example of FIG. 1, system 10 includes a medical device 16, which may be coupled to lead 28. In examples, medical device 16 is coupled to lead 18, lead 28, and/or sensor 22. System 10 may also include an external device 24, which is configured to communicate with medical device 16 via, for example, wireless communication. System 10 may include a server 26 which may be one or more servers in a cloud computing environment. Server 26 may be configured to communicate with external device 24 and/or medical device 16 via wireless communication through a network access point (not shown in FIG. 1), and may be collocated with external device 24 or may be located elsewhere, such as in a cloud computing data center.

Medical device 16 may generally operate as a therapy device that delivers neurostimulation (e.g., electrical stimulation in the example of FIG. 1) to, for example, a target tissue site proximate a spinal nerve, a sacral nerve, a pudendal nerve, dorsal genital nerve, a tibial nerve, a saphenous nerve, an inferior rectal nerve, a perineal nerve, or other pelvic nerves, branches of any of the aforementioned nerves, roots of any of the aforementioned nerves, ganglia of any of the aforementioned nerves, or plexus of any of the aforementioned nerves. Medical device 16 may provide electrical stimulation to patient 14 by generating and delivering a programmable electrical stimulation signal (e.g., a stimulation signal, and/or a signal in the form of electrical pulses or an electrical waveform) to a target a therapy site near lead 28, such as a therapy site near one or more electrodes 30 (“electrodes 30”) supported by and disposed proximal to a distal end 32 of lead 28 (“lead distal end 32”). In examples, electrodes 30 include a plurality of electrodes.

In the example of FIG. 1, electrodes 30 includes electrode 34, electrode 36, electrode 38, and electrode 40. Electrodes 30 may include any number of electrodes in other examples. Electrode 34 is a distal-most electrode of lead 28, and is supported by lead 28 such that electrode 34 is positioned between lead distal end 37 and every other electrode in electrodes 30. Lead 28 may be a sacral lead configured to extend within a sacral foramen of patient 14. Lead 28 may be configured to position one or more of electrodes 30 within or ventral to the sacral foramen. In examples, lead 28 is a sacral lead comprising a sacral lead system 15. Lead 28 may be configured to stimulate a sacral nerve of patient 14 using one or more of electrodes 30 as a stimulation electrode and sense an evoked signal generated by patient 14 using one or more of electrodes 30 as a sensing electrode. Lead 28 may be configured to position the stimulation electrode within or ventral to a foramen of a sacrum of patient 14 and position the sensing electrode dorsal to an anterior opening of the foramen and intracorporeal to the patient. Lead 28 may be configured such that any one electrode (e.g., electrode 34, electrode 36, electrode 38, or electrode 40) may be used as both a stimulation electrode and a sensing electrode. For example, any one electrode may be used to deliver a stimulation signal, and the same any one electrode may be used to receive an evoked signal generated by patient 14 in response to the stimulation signal.

In some examples, electrodes 30 may comprise a single electrode and an external ground, or be configured for use with one electrode and an external ground, e.g., such as may be used for peripheral nerve evaluation (PNE).

In examples, medical device 16 is extracorporeal to patient 14. In some examples, medical device 16 is surgically implanted in patient 14 at a suitable location within patient 14, such as near the pelvis. Medical device 16 may have a biocompatible housing, which may be formed from titanium, stainless steel, a liquid crystal polymer, or the like. The proximal ends of leads 18, 20, and 28 may be both electrically and mechanically coupled to medical device 16 either directly or indirectly, e.g., via respective lead extensions. Lead 18 may include one or more electrodes such as electrode 19A, and lead 20 may include one or more electrodes such as electrode 21A. Electrical conductors disposed within the lead bodies of leads 18, 20, and 28 may electrically connect their respective electrodes to sensing circuitry and a stimulation circuitry (e.g., a stimulation generator) within medical device 16. In examples, electrodes 19 and 21 may be positioned for sensing an impedance of bladder 12, which may increase as the volume of urine within bladder 12 increases. In some examples, system 10 may include other sensors such as additional electrodes, a strain gauge, one or more accelerometers, ultrasound sensors, optical sensors, and/or other sensors. The sensors may be configured to gather information relating to the patient, such as detect contractions of bladder 12, pressure or volume of bladder 12, or any other indication of the fill cycle of bladder 12 and/or possible bladder dysfunctional states. system 10 may use sensors other than electrodes 19 and 21 for sensing information relating to the patient, such as bladder volume, cardiac response, chemical responses, or the like. A stimulation-evoked signal and/or a composite stimulation evoked signal may include a cardiac signal and/or a chemical signal, and the sensors may include a cardiac sensor and/or a chemical sensor. System 10 may use the sensor data for determining stimulation program settings for patient 14. In examples, medical device 16 communicates sensed data to server 26 (e.g., through external device 24).

In some examples, external device 24 may collect user input identifying a voiding event, perceived level of fullness, or any other indication of an event associated with the patient. The user input may be in the form of a voiding journal analyzed by external device 24, medical device 16 or server 26, or individual user inputs associated with respective voiding events, leakage, or any other event related to the patient. External device 24 may provide this user input to server 26.

One or more of leads 18, 20, and 28 may be connected to medical device 16 and surgically or percutaneously tunneled to place one or more electrodes at a desired target therapy site, such as a target therapy site proximate a spinal (e.g., sacral) or pudendal nerve. For example, lead 28 may be positioned such that electrodes 30 deliver electrical stimulation to a sacral nerve to reduce a frequency and/or magnitude of contractions of bladder 12. Leads 18 and 20 may be placed proximate to an exterior surface of the wall of bladder 12 at first and second locations, respectively. In other examples, medical device 16 may be coupled to more than one lead that includes electrodes for delivery of electrical stimulation to different stimulation sites within patient 14, e.g., to target different nerves. In certain embodiments, electrical stimulation will be provided below or at sensory threshold of the patient, but sometimes, in order to evoke or maintain a certain physiological response (i.e. composite signal), the stimulation may be provided above the sensory threshold. In some examples, medical device 16 and/or external device 24 may cause one or more of electrodes 30 to deliver one or more electrical stimulation signals having non-equal pulse amplitudes, non-equal pulse durations, non-equal polarities, and/or non-equal pulse frequencies. In some examples, medical device 16 and/or external device 24 may cause one or more of electrodes 30 to deliver a plurality of electrical stimulation signals according to a known and/or predetermined progression of parameters, e.g., in a “sweep” such as an amplitude or frequency sweep, or the like, alone or in any combination.

External device 24 may be a computing device. In some examples, external device 24 is a clinician programmer or patient programmer. In examples, external device 24 is a device configured for inputting information relating to a patient. External device 24 may be configured to allow a clinician or other user to interact with external device 24 to communicate with medical device 16 and/or server 26. The clinician or other user may interact with external device 24 to retrieve physiological or diagnostic information from medical device 16, program medical device 16 for the generation and delivery of therapy to patient 14, may input information relating to patient 14, and/or other reasons. In some examples, patient 14 may be prompted by external device 24 to provide input related to their medication, lifestyle, quality of life, and other inputs. In some examples, external device 24 is configured to provide a notification to patient 14 when the electrical stimulation is being delivered, and/or notify patient 14 of the prospective termination of the electrical stimulation. In certain embodiments, electrical stimulation will be provided below or at sensory threshold of the patient, but sometimes, in order to evoke or maintain a certain physiological response (i.e. composite signal), the stimulation may be provided above the sensory threshold.

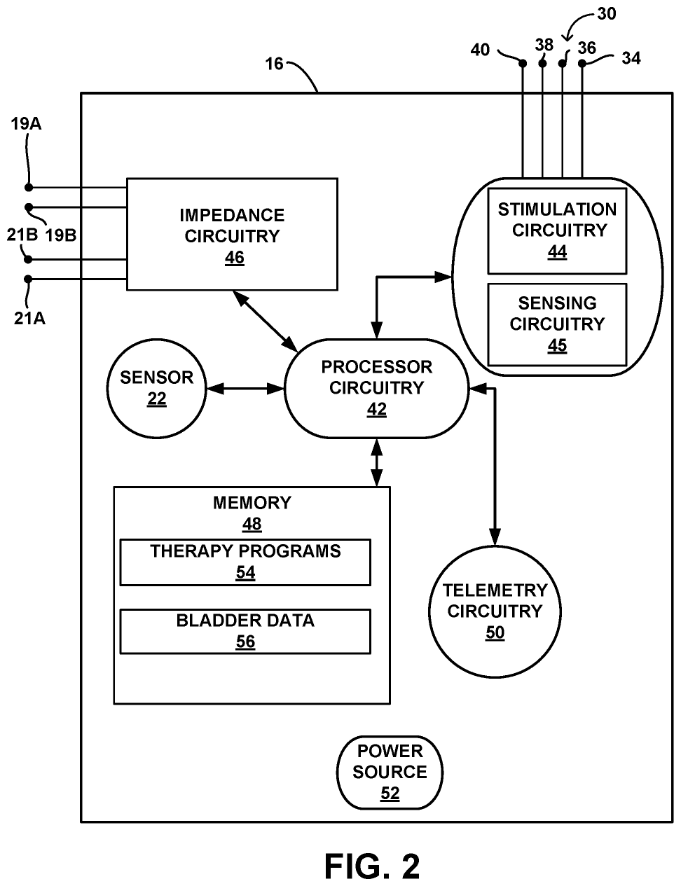

FIG. 2 is a block diagram illustrating an example configuration of an example medical device 16 which may be utilized in the system of FIG. 1. As illustrated in FIG. 2, medical device 16 may include sensor 22, processor circuitry 42, stimulation circuitry 44, sensing circuitry 45, impedance circuitry 46, memory 48, telemetry circuitry 50, and power source 52. In other examples, medical device 16 may include a greater or fewer number of components. Processor circuitry 42 may be configured to identify changes to a physiological state of patient 14 that are relevant to desired changes in neurostimulation based on, for example, a biomarker sensed by a sensor external to medical device 16 (e.g., sensor 22). Memory 48 may store therapy programs 54 that specify stimulation parameter values for the electrical stimulation provided by medical device 16, and may store information relating to determining and using physiological markers, information relating to physiological cycles and/or dysfunctional states, or any other information. In examples, memory 48 stores bladder data 56 associated with physiological events of patient 14. Medical device 16 may provide some or all of bladder data 56 to external device 24 or server 26.

Generally, stimulation circuitry 44 generates and delivers electrical stimulation under the control of processor circuitry 42. Processor circuitry 42 may be configured to access memory 48 to load one of therapy programs 54 to stimulation circuitry 44 for delivering the electrical stimulation to patient 14. A clinician or patient 14 may select a particular one of therapy programs 54 from a list using a programming device, such as external device 24 or a clinician programmer. Processor circuitry 42 may receive the selection via telemetry circuitry 50. Stimulation circuitry 44 delivers the electrical stimulation to patient 14 according to the selected program for an extended period of time, such as minutes, hours, days, weeks, or until patient 14 or a clinician manually stops or changes the program.

Generally, sensing circuitry 45 receives a signal indicative of an evoked signal generated by patient 14 in response to electrical stimulation (e.g., delivered by stimulation circuitry 44). The evoked signal may be indicative of certain aspects of the electrical stimulation therapy delivery, such as the positioning of sacral lead 28 within patient 14. The signal indicative of the evoked signal and received by sensing circuitry 45, or a lack thereof, may indicate that an altered placement of the medical lead within the patient might lead to improved therapies. In examples, sensing circuitry 45 is configured to provide an output indicative of the evoked signal. In examples, the output indicative of the evoked signal is viewable by a clinician.

Impedance circuitry 46 may be configured to communicate with and/or utilize electrodes 19A, 19B, 21A, and/or 21B to detect a physiological state of bladder 12. In examples, impedance circuitry 46 may be configured to communicate with and/or utilize electrodes 34, 36, 38, 40. Power source 52 delivers operating power to the components of medical device 16. Power source 52 may include a battery (e.g., a rechargeable battery) and a power generation circuit configured to produce operating power to medical device 16 and/or other components of system 10 (FIG. 1).

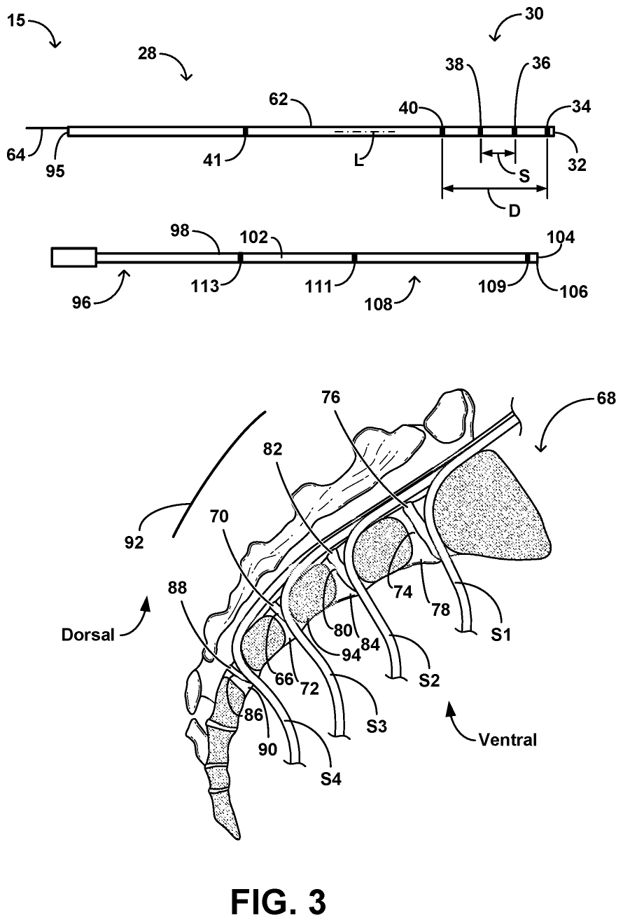

FIG. 3 is a schematic illustration of a sacral lead system 15 including a sacral lead 28. Sacral lead 28 includes a lead body 62 defining a lead distal end 32. Sacral lead 28 supports (e.g., mechanically supports) electrodes 30 including electrode 34, electrode 36, electrode 38, and electrode 40. In examples, electrodes 30 include a return electrode 41. Sacral lead 28 may include one or more conductors 64 configured to electrically connect electrodes 30 to a medical device, such as medical device 16 or another device. In examples, the one or more conductors 64 include a plurality of individual conductors with each individual conductor connected to one of electrode 34, electrode 36, electrode 38, or electrode 40. In examples, each individual conductor is electrically isolated from every other individual conductor in the plurality.

Sacral lead 28 is configured to insert into a sacral foramen 66 of a sacrum 68 of a patient (e.g., patient 14 (FIG. 1)). Sacral lead 28 is configured such that lead body 62 may extend through sacral foramen 66 between posterior opening 70 and anterior opening 72. Sacral lead 28 may be configured to extend from a position dorsal to sacrum 68 to a position ventral to sacrum 68 when lead body 62 inserts into sacral foramen 66. In examples, sacral lead 28 is configured such that lead distal end 32 is ventral to anterior opening 72 when lead body 62 inserts into sacral foramen 66. Sacral lead 28 is configured to position at least one or electrodes 30 proximate a sacral nerve (e.g., one of S1, S2, S3, or S4) when lead body 62 inserts into sacral foramen 66. In examples, sacral lead 28 is configured to position at least one or electrodes 30 proximate sacral nerve S3 extending ventrally from sacral foramen 66 through anterior opening 72.

In addition to sacral foramen 66, sacrum 68 may further include sacral foramen 74 between posterior opening 76 and anterior opening 78, sacral foramen 80 between posterior opening 82 and anterior opening 84, and sacral foramen 86 between posterior opening 88 and anterior opening 90. Although the discussion below mainly refers to sacral foramen 66, posterior opening 70, anterior opening 72, and sacral nerve S3 for illustration, the techniques and configurations described may apply in the same manner to any of sacral foramen 74, posterior opening 76, anterior opening 78, and sacral nerve S1, and/or sacral foramen 80, posterior opening 82, anterior opening 84, and sacral nerve S2, and/or sacral foramen 86, posterior opening 88, anterior opening 90, and sacral nerve S4.

Sacral lead 28 is configured to stimulate sacral nerve S3 when lead body 62 inserts into (e.g., extends through) sacral foramen 66. Sacral lead 28 may be configured to stimulate sacral nerve S3 by delivering a stimulation signal when lead body 62 inserts into sacral foramen 66. In examples, sacral lead 28 is configured to use one or more of electrodes 30 as a stimulation electrode to deliver the stimulation signal. Further, sacral lead 28 is configured to use one or more of electrodes 30 as a sensing electrode to sense an evoked signal generated by patient 14 in response to the stimulation signal. Sacral lead 28 may be configured to use any of electrodes 30 as the stimulation electrode and any of electrodes 30 as the sensing electrode. Further, sacral lead 28 may be configured to use any combination of electrodes 30 as the stimulation electrode and any combination of electrodes 30 as the sensing electrode. In examples, sacral lead 28 is configured to use at least a first electrode (e.g., electrode 34) as the stimulation electrode and use at least a second electrode (e.g., electrode 40) as the sensing electrode. In examples, the second electrode is proximal to the first electrode when lead body 62 inserts into sacral foramen 66. In examples, the second electrode is distal to the first electrode when lead body 62 inserts into sacral foramen 66.

Sacral lead 28 is configured to position electrodes 30 intracorporeal (e.g., below skin 92) to patient 14 when sacral lead 28 positions at least one of electrodes 30 within or ventral (e.g., distal) to sacral foramen 66. In examples, sacral lead 28 is configured to position electrodes 30 intracorporeal to patient 14 when sacral lead 28 positions at least one of electrodes 30 proximate anterior opening 72. In examples, sacral lead 28 defines a displacement D over lead body 62 between a distal-most electrode (e.g., electrode 34) and a proximal-most electrode (e.g., electrode 40). In examples, sacral lead 28 defines the displacement D over lead body 62 between the distal-most electrode and return electrode 41. Sacral lead 28 may be configured to define the displacement D such that electrodes 30 are intracorporeal to patient 14 when sacral lead 28 positions at least one of electrodes 30 proximate anterior opening 72. Hence, sacral lead 28 is configured to extend through posterior opening 70 to stimulate sacral nerve S3 using a stimulation electrode (e.g., one or more of electrodes 30), and configured to sense an evoked signal generated in response to the stimulation signal using a sensing electrode (e.g., one or more of electrodes 30), with the sensing electrode intracorporeal to the patient (e.g., patient 14 (FIG. 1)). In examples, the displacement D is less than about 15 mm.

As used herein, a given electrode within electrodes 30 may be proximate anterior opening 72 when the given electrode is closer to anterior opening 72 than posterior opening 70. In some examples, the given electrode is proximate anterior opening 72 when the given electrode is within sacral foramen 66 and closer to anterior opening 72 than posterior opening 70. In some examples, the given electrode is proximate anterior opening 72 when some portion or substantially all of the given electrode is ventral to anterior opening 72 while another electrode within electrodes 30 and adjacent the given electrode is dorsal to anterior opening 72. In examples, sacrum 68 includes an anterior edge 94 surrounding anterior opening 72, and the given electrode is proximate anterior opening 72 when the given electrode is ventral to posterior opening 70 and closer to anterior edge 94 than any other electrode within electrodes 30. The given electrode may be dorsal to anterior edge 94, ventral to anterior edge 94, or substantially even with anterior edge 94 when the given electrode is proximate anterior opening 72.

In examples, lead body 62 includes a lead opening 95 opening to an internal lumen (not shown) defined by lead body 62 and extending over some length of lead body 62. Lead opening 95 and the internal lumen may be configured to receive a stylet or other elongated body. For example, lead opening 95 and the internal lumen may be configured to allow a clinician to insert the stylet within the internal lumen to guide sacral lead 28 during an implantation procedure. The internal lumen may be configured to receive a force (e.g., in a distal direction) from the stylet and transmit the force to lead body 62. In examples, lead body 62 is configured to flex and/or curve based on forces received from an inserted stylet. For example, lead opening 95 may be configured to receive a stylet defining a curvature, and lead body 62 may be configured to bend to define a similar curvature to the curved stylet as the curved stylet is inserted within the internal lumen.

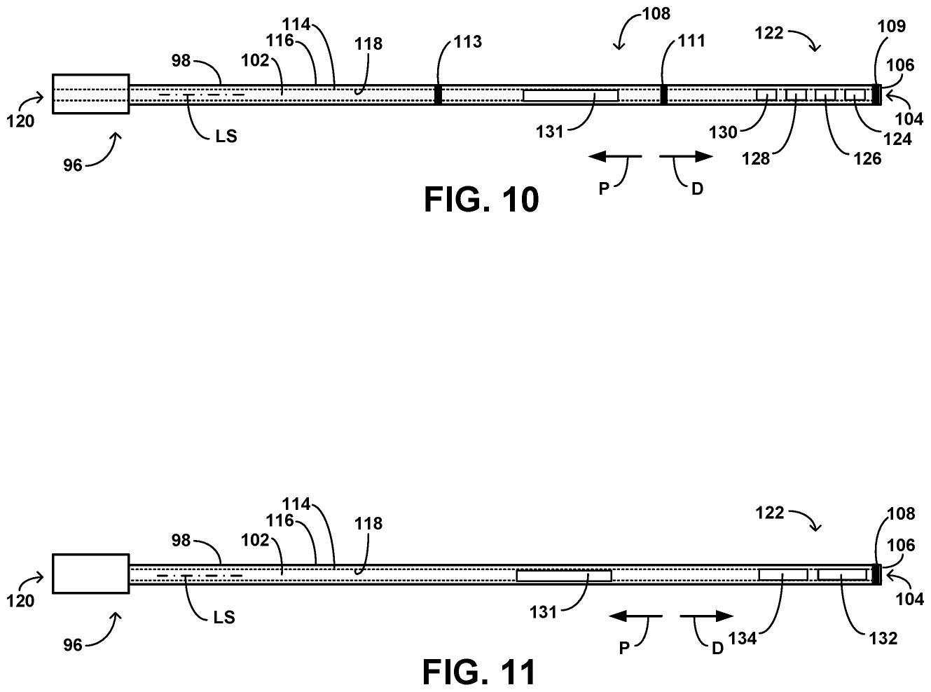

In some examples, sacral lead system 15 may include a sheath 96 (e.g., an introducer sheath) configured to guide sacral lead 28 when sacral lead 28 inserts into sacral foramen 66. Sheath 96 may include a sheath body 98 defining a distal end 106 of sheath body 98 (“sheath distal end 106”). Sheath body 98 may define an inner lumen 102 (“sheath lumen 102”) opening to a sheath opening 104 at sheath distal end 106 (“sheath distal opening 104”). Sheath body 98 may be configured to extend through posterior opening 70 such that sheath distal end 106 is positioned within sacral foramen 66. The sheath body may be configured to position the sheath distal end 106 ventral to anterior opening 72. Sacral lead 28 may be configured such that lead body 62 may be translated through sheath lumen 102 and through sheath distal opening 104 when sheath body 98 extends through posterior opening 70. In examples, sheath 96 includes one or more sheath markers 108, such as sheath marker 109, sheath marker 111, and/or sheath marker 113. configured to be visible by an imaging modality (e.g., a fluoroscope) while within the patient. Sheath 96 may be configured such that a location of sheath distal end 106 and/or other portions of sheath 96 (e.g., one or more of windows 122 (FIG. 10)) within the patient may be ascertained by a clinician based on an observed image of sheath markers 108. For example, sheath 96 may be configured such that one of more of sheath markers 108 are located on sheath 96 (e.g., sheath wall 114 (FIGS. 10, 11)) at a defined distance from or substantially at sheath distal end 106 and/or another portion of sheath 96.

In examples, at least one of sheath markers 108 (e.g., sheath marker 109, sheath marker 111, and/or sheath marker 113) is configured to be imaged by an imaging modality configured to image one or more anatomical features of a patient. The at least one sheath marker may be configured such that an image of the one or more anatomical features and the at least one sheath marker may be utilized (e.g., by a clinician, and/or processor circuity 42) to define a displacement between the at least one sheath marker and at least one of the one or more anatomical features of the patient.

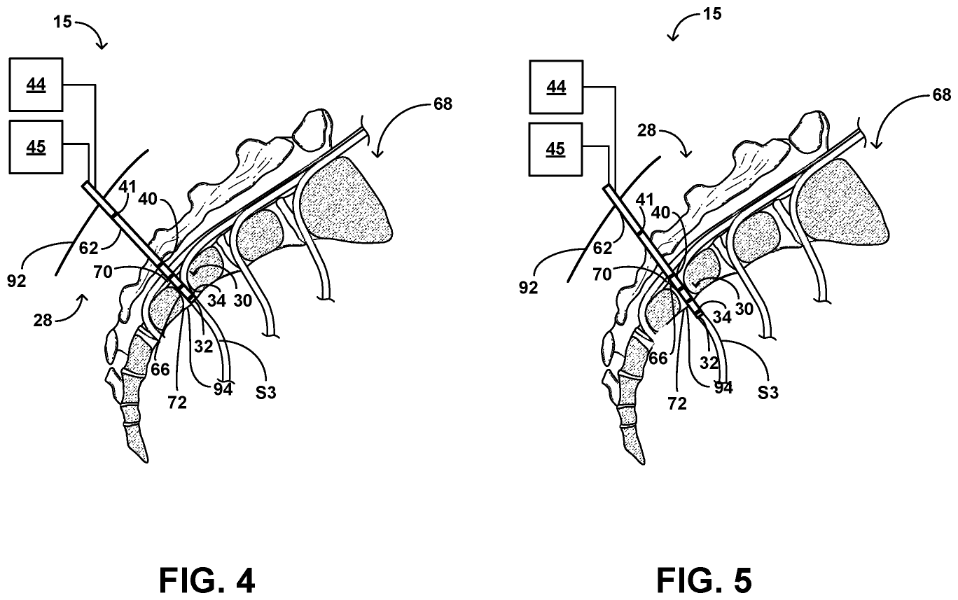

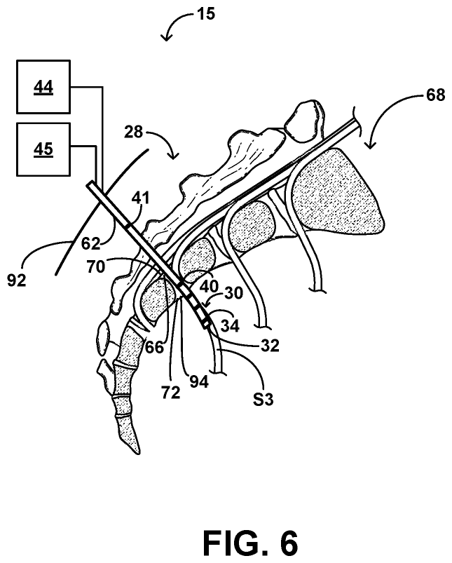

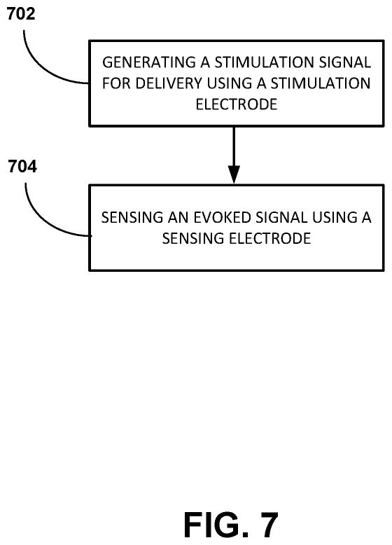

Sacral lead 28 may be configured to enable a technique for positioning sacral lead 28 within a patient using a stimulation signal from at least one stimulation electrode and an evoked signal sensed by at least one sensing electrode. In examples, the technique may include initially positioning the at least one stimulation electrode within or ventral to sacral foramen 66 of the patient and positioning the at least one sensing electrode (e.g., a distal-most electrode) in a plurality of positions successively more ventral based on the evoked signals sensed by sacral lead 28 as sacral lead 28 is inserted. The evoked signal may be indicative of the stimulation received by sacral nerve S3 from electrodes 30 as sacral lead 28 is inserted, such that sacral lead 28 may be positioned to increase and/or optimize the stimulation. The techniques may result in a placement of sacral lead 28 which increases the effectiveness of delivered therapy for the patient, reduces the operating power required by the sacral lead during operation, and/or increases the likelihood of successful placement. Various stages of the implantation technique are discussed and illustrated with reference to FIG. 4, FIG. 5, FIG. 6, and FIG. 7. In some examples, a single electrode of electrodes 30 (e.g., electrode 34, electrode 36, electrode 38, or electrode 40) may be used as both the at least one stimulation electrode and the at least one sensing electrode.

Although the following discusses the use of electrode 34 as an example stimulation electrode for the purpose of explanation, any of one or more of electrodes 30 (e.g., any one or more of electrode 34, electrode 36, electrode 38, and/or electrode 40) may be used in the manner described using electrode 34 in the following or elsewhere within this disclosure. Further, although the following may discuss the use of a specific electrode of electrodes 30 (e.g., any one or more of electrode 34, electrode 36, electrode 38, and/or electrode 40) as an example sensing electrode for the purpose of explanation, any of one or more of electrodes 30 (e.g., any one or more of electrode 34, electrode 36, electrode 38, and/or electrode 40) may be used in the manner described for the sensing electrode in the following or elsewhere in this disclosure.

Further, although the following may discuss using a displacement of some portion of sacral lead 28 (e.g., lead body 62) to position, displace, and/or locate a particular electrode (e.g., any of electrode 34, electrode 36, electrode 38, or electrode 40) when or subsequent to the use of the particular electrode as a stimulation and/or sensing electrode, in examples, instead of or in addition to using a displacement of some portion of sacral lead 28, processing circuitry of the system 10 may cause the positioning, displacement, and/or locating of the stimulation electrode and/or a sensing electrode as lead body 62 remains substantially stationary relative to some portion of the body of patient 14. For example, the processing circuitry may be configured to initially use a first electrode (e.g., electrode 36) at a first location on lead body 62 as the stimulation electrode. The processing circuitry may be configured to subsequently use a second electrode (e.g., electrode 34) at a second location on lead body 62 as the stimulation electrode in order to position and/or displace the stimulation electrode. The processing circuitry may be configured to initially use the first electrode (e.g., electrode 36) at the first location as the sensing electrode. The processing circuitry may be configured to subsequently use the second electrode (e.g., electrode 34) at the second location as the sensing electrode in order to position and/or displace the sensing electrode. Hence, in some examples, the at least one stimulation electrode and/or at least one sensing electrode may be positioned, located, and/or displaced using the processing circuitry of system 10 as lead body 62 remains substantially stationary relative to some portion (e.g., sacral foramen 66, sacral foramen 74, sacral foramen 80, and/or sacral foramen 86) of the body of patient 14.

As illustrated in FIG. 4, the techniques may include extending lead body 62 through posterior opening 70 to position electrode 34 (e.g., the distal-most electrode of electrodes 30) at a first position within or ventral to sacral foramen 66. The techniques include positioning at least one other electrode of electrodes 30 (e.g., electrode 40) dorsal to anterior opening 72 and intracorporeal to the patient when electrode 34 is within or ventral to sacral foramen 66. In examples, the first position places electrode 34 proximate anterior opening 72. The first position may place electrode 34 proximate sacral nerve S3 exiting anterior opening 72. In some examples, the first position places electrode 34 substantially at anterior edge 94.

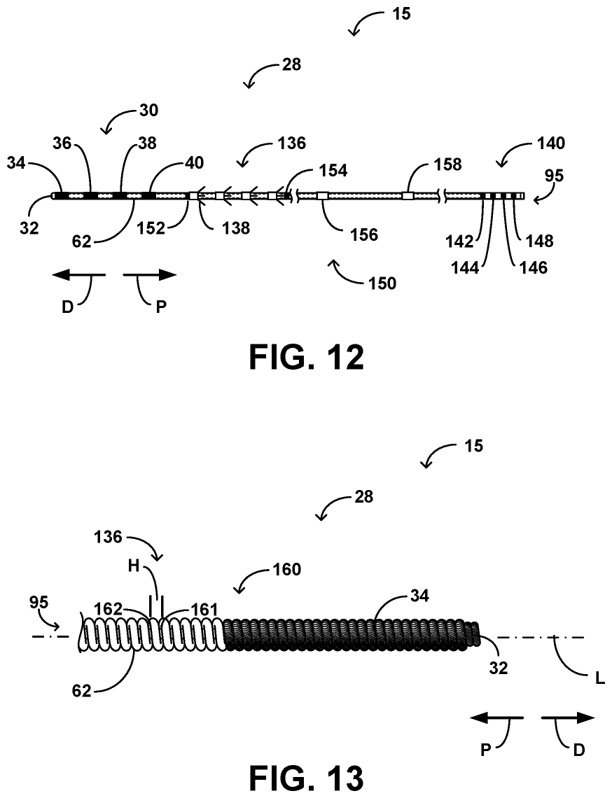

The techniques may include inserting lead body 62 within sacral foramen 66 using sheath 96 (FIG. 3). In examples, sheath 96 is configured to substantially cover a fixation element (e.g., fixation element 136 (FIG. 12)) during the insertion. In examples, sheath 96 is initially inserted within sacral foramen 66. In some examples, sheath distal end 106 may be ventral to posterior opening 70. In examples, one or more of sheath markers 108 indicates when sheath distal end 106 is dorsal to, within, or ventral to posterior opening 70. Lead body 62 may translate through sheath lumen 102 and sheath distal opening 104 to position electrode 34 at the initial position within or ventral to sacral foramen 66.

Sacral lead 28 may deliver a first stimulation signal via electrodes 30 when electrode 34 is in the first position. The technique may include generating the first stimulation signal using stimulation circuitry 44 operably connected to sacral lead 28 and delivering the first stimulation signal to sacral lead 28. Sacral lead 28 may deliver the first stimulation signal using any of electrodes 30, and in any combination, when electrode 34 is in the first position. In examples, sacral lead 28 delivers the first stimulation signal using at least electrode 34 when electrode 34 is in the first position.

The technique may include using sacral lead 28 to receive a first evoked signal generated by the patient in response to the first stimulation signal. Sacral lead 28 may use any of electrodes 30 as a sensing electrode to receive the first evoked signal. In examples, sacral lead 28 uses an electrode proximal to electrode 34 on lead body 62 as the sensing electrode to receive the first evoked signal. The techniques may include receiving the first evoked signal by sensing circuitry 45 operably connected to sacral lead 28. In examples, the technique includes providing an output indicative of the first evoked signal using sensing circuitry 45. In some examples, the technique includes using sensing circuitry 45 to provide an output indicative of the first evoked signal and viewable by a clinician.

In examples, the techniques include altering a power of the first stimulation signal using stimulation circuitry 44 and delivering the altered first stimulation signal via electrodes 30 with electrode 34 is in the first position. The techniques may include using sacral lead 28 to receive an additional first evoked signal generated by the patient, where the additional first evoked signal is generated in response to the altered first stimulation signal. Sensing circuitry 45 may receive the additional first evoked signal. In examples, the techniques include providing an output indicative of the additional first evoked signal using sensing circuitry 45. Sensing circuitry 45 may provide an output indicative of the additional first evoked signal and viewable by a clinician.

The techniques may include further altering the power of the first stimulation signal and causing sacral lead 28 to deliver further stimulation signals using electrodes 30 when electrode 34 is in the first position. The techniques may include receiving other evoked signals generated in response to the further stimulation signals. In examples, the techniques include evaluating the first evoked signal, the additional evoked signal, and/or the other evoked signals generated in response to the further stimulation signals. The techniques may include displacing electrode 34 based on the evaluation. Hence, the techniques may include evaluating an evoked response to evaluate a current position of sacral lead 28 within the patient, and altering a position of sacral lead 28 within the patient based on the evoked response.

As illustrated in FIG. 5, the techniques may include using sacral lead 28 to displace electrode 34 to a second position ventral to the first position (e.g., ventral relative to anterior opening 72). Sacral lead 28 may deliver a second stimulation signal via electrodes 30 when electrode 34 is in the second position. Stimulation circuitry 44 may generate the second stimulation signal and deliver the second stimulation signal to sacral lead 28. Sacral lead 28 may deliver the second stimulation signal using any of electrodes 30, and in any combination, when electrode 34 is in the second position. The technique may include using sacral lead 28 to receive a second evoked signal generated by the patient in response to the second stimulation signal. Sacral lead 28 may use any of electrodes 30 as a sensing electrode to receive the second evoked signal. Sensing circuitry 45 may receive the second evoked signal. Sensing circuitry 45 may provide an output indicative of the second evoked signal. In examples, sensing circuitry 45 provides an output indicative of the second evoked signal and viewable by a clinician.

The techniques may include altering a power of the second stimulation signal using stimulation circuitry 44, delivering the altered second stimulation signal via electrodes 30 with electrode 34 is in the second position, and receiving an additional second evoked signal using lead 28. Sensing circuitry 45 may receive the additional second evoked signal and may provide an output indicative of the additional second evoked signal. The output indicative of the additional second evoked signal may be viewable by a clinician. The technique may include further altering the power of the second stimulation signal and causing sacral lead 28 to deliver further stimulation signals using electrodes 30 when electrode 34 is in the second position, and receiving further evoked signals generated in response to further stimulation signals with electrode 34 at the second position. The technique may include evaluating the second evoked signal, the additional second evoked signal, and/or the further evoked signals with electrode 34 at the second position. The technique may include displacing electrode 34 from the second position based on the evaluation.

The techniques may include continuing to displace electrode 34 to successively more ventral positions relative to anterior opening 72 and continuing to cause lead 28 to issue stimulation signals at each successive position. The technique may include altering a power of the stimulation signal at each successive position and receiving evoked signals generated by the patient in response to the stimulation signals at each successive position. In examples, the technique includes establishing a final position of electrode 34 and lead 28 relative to anterior opening 72 based on the evoked signals received. For example, the technique may include displacing electrode 34 and lead 28 relative to anterior opening 72 until electrode 34 and lead 28 substantially establish a position similar to that illustrated at FIG. 6. In FIG. 6, electrode 34 has established a position ventral to both the first position of FIG. 4 and the second position of FIG. 5. In some examples, lead body 62 is configured to bend and/or flex as electrode 34 is displaced ventrally. Lead body 62 may be configured to bend and/or flex to substantially follow a pathway of sacral nerve S3 as sacral nerve S3 exits anterior opening 72.

In examples, the technique includes issuing a stimulation signal using at least one stimulation electrode located ventral to a posterior opening (e.g., posterior opening 70, posterior opening 76, posterior opening 82, or posterior opening 88) of the foramen (e.g., sacral formen 66, sacral foramen 74, sacral foramen 80, or sacral foramen 86), and receiving an evoked signal using at least one sensing electrode located ventral to the posterior opening. The technique may include issuing a stimulation signal using at least one stimulation electrode located ventral to an anterior opening (e.g., anterior opening 72, anterior opening 78, anterior opening 84, or anterior opening 90) of a foramen, and receiving an evoked signal using at least one sensing electrode located ventral to a posterior opening of the foramen. The technique may include issuing a stimulation signal using at least one stimulation electrode located within the foramen, and receiving an evoked signal using at least one sensing electrode located ventral to an anterior opening of the foramen. The technique may include issuing a stimulation signal using at least one stimulation electrode located ventral to a posterior opening of the foramen, and receiving an evoked signal using at least one sensing electrode located dorsal to an anterior opening of the foramen. The technique may include issuing a stimulation signal using at least one stimulation electrode located dorsal to an anterior opening of the foramen, and receiving an evoked signal using at least one sensing electrode located ventral to a posterior opening of the foramen. The technique may include issuing a stimulation signal using at least one stimulation electrode located dorsal to an anterior opening of the foramen, and receiving an evoked signal using at least one sensing electrode located dorsal to the anterior opening of the foramen.

FIG. 7 is a flowchart illustrating an example technique for positioning sacral lead 28 within a patient using a stimulation signal from at least one stimulation electrode and an evoked signal sensed by at least one sensing electrode. The at least one stimulation electrode may be one of more stimulation electrodes defined by one or more electrodes of the lead. The at least one sensing electrode may be one of more sensing electrodes defined by the one or more electrodes of the lead. In examples, at least one electrode in the one or more electrodes may acts as the at least one stimulation electrode and the at least one sensing electrode. The technique may be similar to that discussed with reference to FIG. 4, FIG. 5, and FIG. 6.

The technique includes generating a stimulation signal (e.g., using stimulation circuitry 44) for delivery using the at least one stimulation electrode supported by sacral lead 28 (702). The technique may include positioning the at least one stimulation electrode within or ventral to sacral foramen 66 of the patient and positioning the at least one sensing electrode dorsal to anterior opening 72 of sacral foramen 66 and intracorporeal to the patient 14. The technique may include delivering the stimulation signal using the at least one stimulation electrode. The technique further includes sensing an evoked signal using the at least one sensing electrode (704). The technique may include receiving a signal indicative of the evoked signal using sensing circuitry 45. In examples, the technique may include using sensing circuitry 45 to provide an output indicative of the evoked signal. The technique may include using sacral lead 28 and/or processing circuitry of system 10 to displace the at least one stimulation electrode subsequent to receiving the evoked signal (e.g., based on the evoked signal). In examples, the technique includes using sacral lead 28 to displace a distal-most electrode and/or other electrodes of the one or more electrodes in a plurality of positions successively more ventral based on the evoked signals sensed by sacral lead 28 as sacral lead 28 is inserted and/or the processing circuitry of system 10 displaces the distal-most electrode and/or other electrodes of the one or more electrodes.

The processing circuitry of system 10 may include and/or control stimulation circuitry configured to deliver stimulation energy with stimulation parameters specified by one or more stimulation parameter settings stored on a storage device and/or configured to collect stimulation-evoked signals and/or accompanying signals pertaining to the stored stimulation parameter settings. The processing circuitry may collect this stimulation-evoked signal and/or accompanying signals information by receiving the information via sensing circuitry and/or directly from sensors (e.g., electrodes 19, 21, 30). The processing circuitry may include and/or control the sensing circuitry. The processing circuitry may also include and/control stimulation generation circuitry to test different parameter settings and record one or more corresponding stimulation-evoked signals and/or accompanying signals for each selected combination, and test different parameter settings as they compare to one or more sensed stimulation-evoked signals and/or accompanying signals.

For example, the processing circuitry may direct stimulation generation circuitry (e.g., stimulation circuitry 44) to deliver stimulation via a particular cycling and a signal unit may collect the corresponding stimulation-evoked signal data from telemetry circuitry. The stimulation-evoked signal data for this test may be stored in the storage device. The processing circuitry may adjust the previously tested cycling of the stimulation delivered via the electrode combination to a different cycling and collect the corresponding stimulation-evoked signal data from sensors and the sensing circuitry in response to stimulation with the adjusted cycling. The stimulation-evoked signal data received for the stimulation at the changed stimulation parameter, such as cycling, may be saved in the storage device and may be output to a user. The processing circuitry may continue to shift the cycling by either increasing or decreasing the cycling frequency and/or cycling duty cycle, and record the respective stimulation-evoked signal data which is stored on the storage device, and information based on the stimulation-evoked signal data may be output to a user. While the example of cycling is provided, processing circuitry may direct stimulation circuitry to step through various incremental settings of other stimulation parameters, such as electrode combination or configuration, electrode polarity, amplitude, pulse width, pulse shape, pulse frequency or pulse rate, or cycling and record respective stimulation-evoked signal data for each stepped value. In one or more examples, the processing circuitry may direct stimulation circuitry to turn on for a certain period of time, and/or to turn off for a period of time, or to turn on at a certain time of day and record the respective stimulation-evoked signal data. The stimulation circuitry may shift more than one stimulation parameter for each test and collect sensed stimulation-evoked signal data for each of the multiple shifted stimulation parameters.

The stimulation generation circuitry may include electrical stimulation circuitry configured to generate electrical stimulation and generates electrical stimulation pulses selected to alleviate symptoms of one or more diseases, disorders or syndromes. Stimulation signals may include a stimulation pulse and/or other forms, such as continuous-time signals (e.g., sine waves) or the like. The electrical stimulation circuitry may reside in an implantable housing, for example of IMD 16. In other examples, the electrical stimulation circuitry may reside in an external medical device housing (e.g., of external device 24), e.g., an external device including the circuitry and functionality of IMD 16 or other devices described herein and configured to directly connect to leads 28, 18, 20. Each of leads 28, 18, 20 may include any number of electrodes 130, 140. The electrodes are configured to deliver the electrical stimulation to the patient. In the example of FIGS. 1, 3-6, 8, 9, 12, 13, each set of electrodes 130, 140 includes for electrodes. Although electrodes 130, 140 are described with eight electrodes, electrodes 130, 140 may have more or fewer electrodes, for example, electrodes 130, 140 may have a single electrode, two electrodes, three electrodes, four electrodes, or five, six, or seven electrodes, or nine or more electrodes. In the examples shown, electrode sets 130 and 140 have the same number of electrodes. In other examples, electrode sets 130 and 140 may have a different number of electrodes from each other. In some examples, the electrodes are arranged in bipolar combinations. A bipolar electrode combination may use electrodes carried by the same lead 28, 18, 20 or different leads. For example, an electrode A of electrodes 130 may be a cathode and an electrode B of electrodes 130 may be an anode, forming a bipolar combination. In other examples, the electrodes may be monopolar. For example, a housing of lead 28, 18, 20 and/or lead 28, 18, 20, or IMD 16, or a ground patch (not shown) may function as the return path for one or more electrodes 130 and/or electrodes 140 in a monopolar configuration. Switching circuitry may include one or more switch arrays, one or more multiplexers, one or more switches (e.g., a switch matrix or other collection of switches), or other electrical circuitry configured to direct stimulation signals from stimulation circuitry 44 to one or more of electrodes 130, 140, or directed sensed signals from one or more of electrodes 130, 140 to sensing circuitry 45. In some examples, one or more of electrodes 130, 140 may be configured to both deliver stimulation signals and sense signals, and switch circuitry may be configured to direct stimulation signals from stimulation circuitry 44 to such electrodes and direct sensed signals, sensed by such electrodes, to sensing circuitry 45. In some examples, each of the electrodes 130, 140 may be associated with respective regulated current source and sink circuitry to selectively and independently configure the electrode to be a regulated cathode or anode. Stimulation circuitry 44 and/or sensing circuitry 45 also may include sensing circuitry to direct electrical signals sensed at one or more of electrodes 130, 140.

As discussed, sacral lead 28 may be configured to define a displacement “D” (FIG. 3) such that electrodes 30 are intracorporeal to patient 14 when sacral lead 28 positions at least one of electrodes 30 proximate anterior opening 72. In examples, sacral lead 28 defines a spacing S (FIG. 3) between adjacent electrodes (e.g., electrode 36 and electrode 38) to defines the displacement D. In some examples, the spacing “S” is substantially uniform between all adjacent electrodes. In other examples, the spacing S is non-uniform, such that lead body defines a first spacing between a first pair of adjacent electrodes (e.g., electrode 34 and electrode 36) and defines a second spacing between a second pair of adjacent electrodes (e.g., electrode 36 and electrode 38), with the first spacing unequal to the second spacing. For example, the first spacing may be less than or equal to 3 mm while the second spacing may be greater than 3 mm. The first spacing and the second spacing may define other displacements in other examples. Lead body 62 may define the various spacings such that one or more of electrodes 30 position as described herein (e.g., within sacral foramen 66 or dorsal to posterior opening 70) when a distal-most electrode is proximate anterior opening 72.

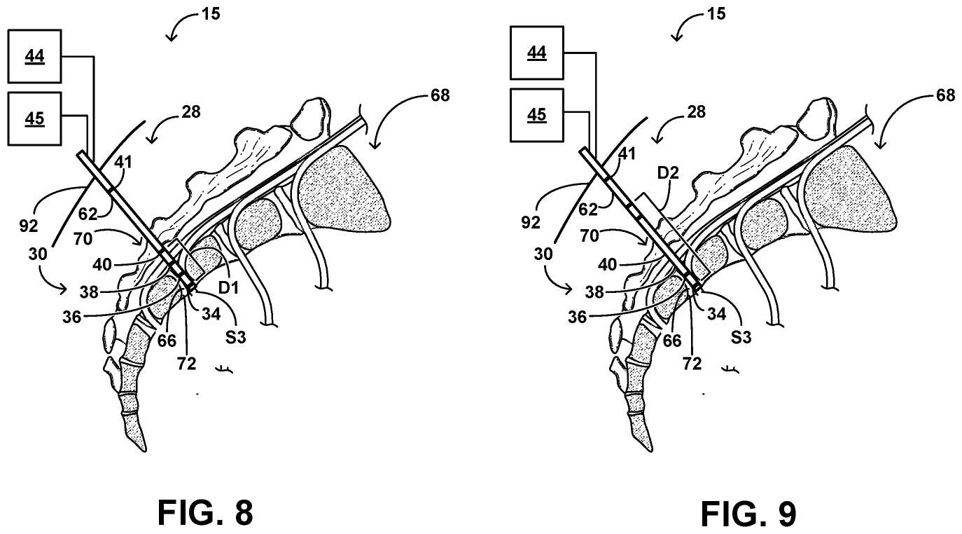

As an example, FIG. 8 illustrates an example sacral lead 28 supporting electrodes 30 spaced such that electrodes 30 define a displacement D1 along a length of lead body 62. Electrodes 30 include distal-most electrode 34, electrode 36, electrode 38, and electrode 40. In the example of FIG. 8, sacral lead 28 (e.g., electrodes 30) is configured to define the displacement D1 such that when a distal-most electrode (electrode 34) is proximate anterior opening 72, all of electrodes 30 (e.g., electrodes 36, 38, 40) may be ventral to posterior opening 70 and/or within sacral foramen 66. Hence, sacral lead 28 may be configured such that when any of electrodes 30 acts as the one or more stimulation electrodes, any of electrodes 30 may act as the one or more sensing electrodes from a position ventral to posterior opening 70 and/or within sacral foramen 66. Sacral lead 28 (e.g., lead body 62) may be configured to define a spacing between each pair of adjacent electrodes (e.g., electrode 34 and electrode 36, electrode 36 and electrode 38, and/or electrode 38 and electrode 40) to define the displacement Dl.

FIG. 9 illustrates an example sacral lead 28 supporting electrodes 30 spaced such that electrodes 30 define a displacement D2 along a length of lead body 62. In the example of FIG. 9, sacral lead 28 (e.g., electrodes 30) is configured to define the displacement D2 such that when a distal-most electrode (electrode 34) is proximate anterior opening 72, one or more of electrodes 30 (e.g., electrodes 38, 40) are dorsal to posterior opening 70. Hence, sacral lead 28 may be configured such that when an electrode within sacral foramen 66 and/or proximate anterior opening 72 (e.g., electrode 34 and/or electrode 36) acts as the stimulation electrode, the one or more electrodes dorsal to posterior opening 70 (e.g., electrode 38 and/or electrode 40) may act as the sensing electrode from a position dorsal to anterior opening 72.

FIGS. 8 and 9 provide examples of the displacement D1 and the displacement D2. As discussed, sacral lead 28 may be configured to provide any displacement D over the one or more electrodes or any spacing S between electrodes of the one or more electrodes. Sacral lead 28 may be configured such that electrodes 30 define at least one stimulation electrode located dorsal to, within, or ventral to a foramen (e.g., sacral foramen 66) when electrodes 30 define at least one sensing electrode dorsal to, within, or ventral to the foramen.

FIG. 10 illustrates an example sheath 96 configured to guide sacral lead 28 when sacral lead 28 inserts into sacral foramen 66. Sheath body 98 includes a wall 114 (“sheath wall 114”) defining an outer surface 116 and an inner surface 118 opposite outer surface 116. Inner surface 118 defines sheath lumen 102 opening to sheath distal opening 104. Sheath lumen 102 may extend to an opening 120 in a proximal portion of sheath body 98 (“sheath proximal opening 120”), such that sheath lumen 102 defines a lumen interior extending from sheath proximal opening 120 to sheath distal opening 104. Sheath 96 may be configured such that, when sheath distal opening 104 is positioned within sacral foramen 66 and/or proximate anterior opening 72 of patient 14 (FIG. 1), sheath proximal opening 120 is extracorporeal to patient 14. Sheath 96 may be configured such that lead body 62 (FIG. 3-6, 8, 9) may extend though sheath proximal opening 120, through sheath lumen 102, and through sheath distal opening 104. Sacral lead 28 may be configured to slidably translate through sheath proximal opening 120, sheath lumen 102, and sheath distal opening 104.

Sheath 96 may be configured to insert into sacral foramen 66 to guide sacral lead 28 to position at least ventral to posterior opening 70 (FIGS. 3-6, 8, 9). Sheath 96 may be configured such that sheath proximal opening 120 may receive lead body 62 when sheath distal opening 104 is ventral to posterior opening 70. Sacral lead 28 may be configured to translate through sheath proximal opening 120 and sheath lumen 102 when sheath distal opening 104 is ventral to posterior opening 70 such that at least lead distal end 32 positions ventral to posterior opening 70. In examples, sheath 96 is configured to position sheath distal opening 104 proximate and/or ventral to anterior opening 72. Sacral lead 28 (e.g., lead body 62) may be configured to place one or more of electrodes 30 ventral to posterior opening 70 when sheath distal opening 104 is positioned ventral to posterior opening 70.

Sheath 96 may be configured to substantially allow or prevent sacral lead 28 from communicating a stimulation signal to tissues and/or a sacral nerve of patient 14 when lead body 62 is positioned within sheath lumen 102. Sheath 96 may be configured such that sheath 96 substantially blocks an electrode within electrodes 30 from communicating the stimulation signal to tissues and/or the sacral nerve when sacral lead 28 is in a first position within sheath lumen 102, and substantially allows the electrode within electrodes 30 to communicate the stimulation signal to tissues and/or the sacral nerve when sacral lead 28 is in a second position within sheath lumen 102. In examples, sacral lead 28 is configured to translate relative to inner surface 118 and within sheath lumen 102 from the first position to the second position (or vice-versa), such that sheath 96 substantially blocks or allows the stimulation signal from the electrode. In examples, when sheath 96 substantially blocks a stimulation signal from an electrode, this may mean that sheath wall 114 attenuates and/or otherwise reduces the stimulation signal emitted by the stimulation electrode prior to the stimulation signal reaching tissues and/or sacral nerves of the patient.

In examples, and as illustrated in FIG. 10, sheath 96 includes one or more windows 122 such as window 124, window 126, window 128, and window 130. Each of windows 122 defines an opening in sheath wall 114 configured to allow a stimulation electrode to emit a stimulation signal from within sheath lumen 102 to an exterior of sheath 96 through the opening when the stimulation electrode is aligned with the window. Each of windows 122 may be configured to allow a sensing electrode to sense an evoked signal through the opening when the sensing electrode is aligned with the window. In examples, windows 122 include a return window 131 configured to allow a return electrode (e.g., electrode 41) to electrode connect with a medium surrounding sheath 96 (e.g., blood of patient 14) when the return electrode is within sheath lumen 102. Lead body 62 is configured to slidably translate within sheath lumen 102 to align the stimulation electrode and/or sensing electrode and a window. Sheath 96 may be configured such that, as lead body 62 translates within sheath lumen 102, windows 122 allow differing combinations of electrodes 30 to transmit stimulation signals to an exterior of sheath 96 and/or sense evoked signals, such that a clinician may evaluate the differing combinations by re-positioning lead body 62 within sheath lumen 102.

For example, lead body 62 may be configured to position within sheath lumen 102 in a first position wherein a distal-most electrode such as electrode 34 (FIGS. 3-6, 8, 9) distal to sheath distal end 106 while one or more sensing electrodes of electrodes 30 (e.g., electrode 36, electrode 38, electrode 40) are proximal to sheath distal end 106 (e.g., displaced in the proximal direction P from sheath distal end 106). Sheath 96 may be configured such that, in the first position, electrode 34 may act as a stimulation electrode to emit a stimulation signal. A sensing electrode may be aligned with a window of windows 122 such that the sensing electrode may sense an evoked signal through the opening defined by the window. Sheath 96 may be configured such that sheath wall 114 substantially blocks sensing electrodes not aligned with a window of windows 122 from receiving the evoked signal. Lead body 62 may be configured to translate distally (e.g., in the distal direction D) within sheath lumen 102 to a second position wherein additional sensing electrodes align with a window of windows 122, such that the additional sensing electrodes may sense an evoked signal through the opening defined by the respective aligned window. Hence, sheath 96 and lead body 62 may be configured such that a clinician may evaluate the efficacy of stimulation signals by re-positioning lead body 62 within sheath lumen 102 to evaluate the evoked signals sensed.

In examples, lead body 62 may be configured to position within sheath lumen 102 in a first position wherein a distal-most electrode such as electrode 34 (FIGS. 3-6, 8, 9) is aligned with window 130 while the remaining electrodes of electrodes 30 (e.g., electrode 36, electrode 38, electrode 40) are proximal to window 130 (e.g., displaced in the proximal direction P from window130). Sheath 96 may be configured such that, in the first position, electrode 34 may act as a stimulation electrode to emit a stimulation signal through the opening defined by window 130 as sheath wall 114 substantially blocks stimulation signals emitted by the remaining electrodes. Lead body 62 may be configured to translate distally (e.g., in the distal direction D) within sheath lumen 102 to a second position wherein electrode 34 aligns with window 128 and electrode 36 aligns with window 130 as the remaining electrodes of electrode 130 remain proximal to window 130. Sheath 96 may be configured such that, in the second position, electrode 34 and electrode 36 may emit a stimulation signal through the openings defined by window 128 and window 130 as sheath wall 114 substantially blocks stimulation signals emitted by the remaining electrodes. Sheath 96 may be configured such that lead body 62 may translate to a third position aligning other electrodes of electrode 30 with a window of windows 122. Hence, sheath 96 and lead body 62 may be configured such that a clinician may evaluate the efficacy of stimulation signals delivered by differing combinations of electrodes 30 by re-positioning lead body 62 within sheath lumen 102 and/or based on any evoked signals.

In examples, sheath 96 is configured such that an individual window (e.g., window 124, window 126, window 128, and/or window 130) within windows 122 is configured to align with a single electrode as lead body 62 translates within sheath lumen 102. In other examples, as illustrated in FIG. 11, windows 122 may include one or windows such as window 132 and/or window 134 configured to align with a plurality of electrodes (e.g., two or more) within electrodes 30 when lead body 62 translates within sheath lumen 102. Sheath 96 may include any combination of windows including windows configured to align with only a single electrode (e.g., windows 124, 126, 128, and/or 130) and windows configured to align with a plurality of windows (e.g., windows 132 and/or 134). Sheath 96 may be configured to define windows 122 to accommodate any distance D and any spacing S (FIGS. 3, 10, 11) of electrodes 30. In some examples, one or more of sheath markers 108 may be at a defined distance and/or substantially aligned with one window of windows 122 such that, for example, an imaged representation of the one or more sheath markers in an image including anatomical features of the patient may be used (e.g., by a clinician or processing circuitry of system 10) to ascertain and/or estimate a position of the one window relative to the anatomical features or relative to one or more of electrodes 130, 140. In some examples, each window of windows 122 includes a separate sheath marker at a defined distance and/or substantially aligned with the each window.

In examples, sheath 96 is configured such that an individual window (e.g., window 124, window 126, window 128, and/or window 130) aligns with only a portion of an electrode comprising one or more electrodes. For example, the electrode may be a ring electrode or some other type of electrode. The individual window may be configured to align with the portion of the electrode as sheath 96 (e.g., sheath wall 114) substantially covers a remainder of the electrode, such that the electrode may emit stimulation signals and/or receive sensed signals substantially in a direction established by the single window. For example, the individual window may be configured such that the electrode may emit stimulation signals and/or receive sensed signals substantially in a first direction when the individual window has a first orientation relative to lead body 62 and/or an anatomical feature of a patient. Sheath 96 may be rotated relative to the electrode (and e.g., lead body 62) to cause the individual window to establish a second orientation relative to lead body 62 and/or an anatomical feature of a patient. The second orientation of the individual window may allow the electrode to emit stimulation signals and/or receive sensed signals substantially in a second direction corresponding to the second orientation. Hence, the individual window may be configured such that rotation of sheath 96 substantially establishes a directionality for the electrode.

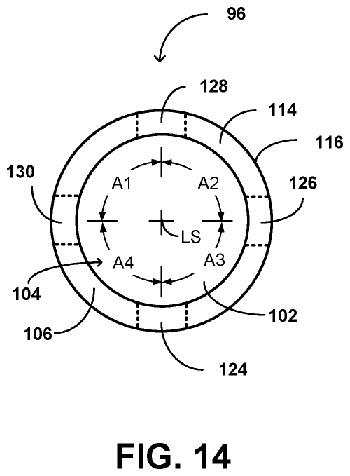

In examples, as illustrated at FIG. 14, windows 122 (e.g., window 124, window 126, window 128, and/or window 130) may be angularly offset from one another around a sheath longitudinal axis LS defined by sheath 96 (e.g., defined by sheath lumen 102). FIG. 14 provides a plan view of sheath distal end 106, with windows 124, 126, 128, 130 illustrated in dashed lines for clarity.

Sheath 96 (e.g., sheath wall 114) may define any angular offset between adjacent windows within windows 122. In examples, at least one window of windows 122 may be angularly offset from one or more of windows 122 adjacent to the at least one window, For example, window 130 may be angularly offset by the angle A1 from window 128. Window 128 may be angularly offset by the angle A2 from window 126. Window 126 may be angularly offset by the angle A3 from window 124. Window 124 may be angularly offset by the angle A4 from window 130.

Windows 122 may be angularly offset (e.g., clocked) around lumen wall 114 and/or exterior surface 116 such that, for example, rotation of sheath 96 relative to lead body 92 causes one of more of windows 122 to align with one or more of electrodes 30. In examples, the rotation of sheath 96 causing the one of more of windows 122 to align with the one or more of electrodes 30 causes sheath wall 114 to additionally shield one or more of electrodes 30. For example, in a first rotational position, sheath 96 may be oriented relative to lead body 92 such that window 130 aligns with electrode 40 (FIGS. 3-6) as sheath wall 114 shields electrode 38, electrode 36, and/or electrode 34. Sheath 96 may be configured such that in a second rotation rotational position relative to lead body 96, such that window 128 aligns with electrode 38 as sheath wall 114 shields electrode 40, electrode 36, and/or electrode 34. Sheath 96 may be configured such that in a third rotation rotational position relative to lead body 96, window 126 aligns with electrode 36 as sheath wall 114 shields electrode 40, electrode 38, and/or electrode 34, and such that in a fourth rotational position relative to lead body 96, window 124 aligns with electrode 34 as sheath wall 114 shields electrode 40, electrode 36, and/or electrode 36.

Sheath 114 may define any angular offset between any of windows 122. In examples, two or more of the angular offsets (e.g., angular offset A1, angular offset A2, angular offset A3, and/or angular offset A4) describe substantially uniform (e.g., substantially equal) angles. In some examples, two or more of the angular offsets (e.g., angular offset A1, angular offset A2, angular offset A3, and/or angular offset A4) describe substantially non-uniform (e.g., substantially unequal) angles.

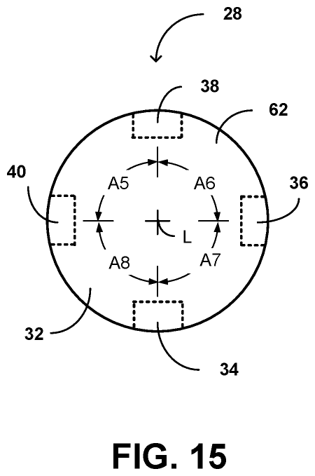

In some examples, as illustrated at FIG. 15, instead of or in addition to sheath 114 defining the angular offsets, sacral lead 28 (e.g., lead body 62) may be configured such that one or more of electrodes 30 define angular offsets. Sacral lead 28 may be configured such that one or more of electrodes 130 define angular offsets around a longitudinal axis L defined by lead body 62. FIG. 15 provides a plan view of lead distal end 32, with electrodes 34, 36, 38, 40 illustrated in dashed lines for clarity.

Electrodes 30 may be angularly offset (e.g., clocked) around sheath body 92 such that, for example, rotation of lead body 92 relative to sheath 96 causes one of more of electrodes 30 to align with one or more of windows 122. In examples, sacral lead 28 is configured such that at least one electrode of electrodes 30 may be angularly offset from one or more of electrodes 30 adjacent to the at least electrode. For example, electrode 40 may be angularly offset by the angle A5 from electrode 38. Electrode 38 may be angularly offset by the angle A6 from electrode 36. Electrode 36 may be angularly offset by the angle A7 from electrode 34. Electrode 34 may be angularly offset by the angle A8 from electrode 40. Any of angular offsets A5, A6, A7, A8 may describe substantially similar angular offsets or substantially different angular offsets from any of angular offsets A1, A2, A3, A4.

In examples, the rotation of lead body 92 causing the one of more of electrodes 30 to align with the one or more of windows 122 may cause sheath wall 114 to additionally shield one or more of electrodes 30. For example, electrodes 30 may be clocked around lead body 92 such that in a first rotational position of lead body 92 relative to sheath 96 such that one of windows 122 is aligned with electrode 40 as shield wall 114 shields electrode 38, electrode 36, and/or electrode 34. Electrodes 30 may be clocked around lead body 92 such that in a second rotational position of lead body 92 relative to sheath 96, one of windows 122 is aligned with electrode 40 as shield wall 114 shields electrode 38, electrode 36, and/or electrode 34. Electrodes 30 may be clocked around lead body 92 such that in a third rotational position of lead body 92 relative to sheath 96, one of windows 122 is aligned with electrode 36 as shield wall 114 shields electrode 40, electrode 38, and/or electrode 34. Electrodes 30 may be clocked around lead body 92 such that in a fourth rotational position of lead body 92 relative to sheath 96, one of windows 122 is aligned with electrode 34 as shield wall 114 shields electrode 40, electrode 38, and/or electrode 36.

FIG. 12 illustrates an example sacral lead 28 including a fixation structure 136 supported (e.g., mechanically supported) by lead body 62. Fixation structure 136 may be configured to resist translation of sacral lead 28 when sacral lead 28 is implanted within patient 14 (FIG. 1). Fixation structure 136 may include one or more fixation elements such as fixation element 138. In examples, fixation structure 136 is configured to offer low or substantially no resistance to proximal and/or distal movements of lead body 62 when lead body 62 is within sheath lumen 102 of sheath 96 (FIGS. 10-11). Fixation structure 136 may be configured to substantially deploy when sheath distal opening 104 is proximal to fixation structure 136 (e.g., when a clinician proximally withdraws sheath 96). For example, fixation element 138 may be resiliently biased to cause a free end of fixation element 138 to spring outward away from lead body 62 when sheath distal opening 104 is proximal to fixation structure 136. Fixation structure 136 may be configured to engage tissue of patient 14 when fixation element 136 deploys. In examples, fixation structure 136 is configured to engage tissue of patient 14 within sacral foramen 66 when one or more of electrodes 30 are proximate and/or ventral to anterior opening 172.

In examples, sacral lead 28 includes one or more connectors 140 operably connected to one or more of electrodes 30. Connectors 140 may be supported (e.g., mechanically supported) by lead body 62. In examples, sacral lead 28 includes one or more conductors operably coupling connectors 140 with electrodes 30. In some examples, sacral lead 28 is configured such that each connector of connectors 140 is operably coupled to one of electrodes 30 by an individual conductor electrically isolated from every other conductor in the one or more conductors. For example, referring to FIG. 12, sacral lead 28 may be configured such that connector 142 is operably connected to electrode 34 through a first conductor (not shown), connector 144 is operably connected to electrode 36 through a second conductor (not shown), connector 146 is operably connected to electrode 38 through a third conductor (not shown), and/or connector 148 is operably connected to electrode 40 through a fourth conductor (not shown). Each of the first conductor, second conductor, third conductor, and fourth conductor may be electrically isolated from every other conductor.

In examples, sacral lead 28 is configured such that connectors 140 may operably couple with stimulation circuitry 44 and/or sensing circuitry 45 (FIGS. 3-6, 8, 9). In examples, sacral lead 28 is configured such that connector 140 may operably couple with an external device configured to operably couple connectors 140 with stimulation circuitry 44 and/or sensing circuitry 45. Connectors 140 may be configured to mechanically mate with a terminal of the external device. Sacral lead 28 may be configured such that an individual connector within connectors 140 may be connected (e.g., by the external device) to stimulation circuitry 44 and/or sensing circuitry 45 independently of another connector within connectors 140. For example, sacral lead 28 may be configured such that connectors 140 operably connects electrode 34 and/or electrode 36 to stimulation circuitry 44 while operably connecting electrode 38 and/or electrode 40 to sensing circuitry 45, or vice-versa. Sacral lead 28 may be configured such that connectors 140 operably connect any combination of electrodes 30 to stimulation circuitry 44 while coupling any other combination or the same combination to sensing circuitry 45. Thus, connectors 140 may be configured such that an external device may substantially dictate whether a given electrode operates as a stimulation electrode, a sensing electrode, or both a stimulation electrode and a sensing electrode. Connectors 140 may be configured such that a clinician (e.g., by providing an input to an external device) may determine whether the given electrode operates as a stimulation electrode, a sensing electrode, or both a stimulation electrode and a sensing electrode.

In examples, lead body 62 supports (e.g., mechanically supports) one or more imaging markers 150 such as marker 152, marker 154, marker 156, and/or marker 158. Markers 152, 154, 156, 158 may be configured to be visible by an imaging modality (e.g., a fluoroscope) while within patient 14 (FIG. 1). Markers 152, 154, 156, 158 may be configured such that a position of lead body 62 within the patient may be ascertained by a clinician based on an observed image of markers 152, 154, 156, 158. In examples, markers 152, 154, 156, 158 are defined on lead body 62 in a specific location relative to electrodes 30, fixation structure 136, another of markers 152, 154, 156, 158 or some other section of lead body 62.

In examples, markers 152, 154, 156, 158 are be configured such that comparison of imaged representations of markers 152, 154, 156, 158 within an image of sacral foramen 66 of patient 14 provides an indication of the position of lead body 62 relative to sacral foramen 66. For example, markers 152, 154, 156, 158 may be configured such that the imaged representations provide an indication of a position of lead body 62 (e.g., distal end 32, electrodes 30, and/or fixation structure 136) relative to posterior opening 170 and/or anterior opening 172 of sacral foramen 66. In some examples, markers 152, 154, 156, 158 are configured such comparison of image representations of markers 152, 154, 156, 158 with an imaged representation of sheath markers 108 (FIGS. 3, 10, 11) provides an indication of the position of lead body 62 relative to sheath 96 within patient 14. In examples, markers 152, 154, 156, 158 (e.g., marker 152) are configured to provide an indication (e.g., by comparison with sheath markers 108) of whether electrodes 30 have extended ventrally beyond sheath distal end 106. In examples, markers 152, 154, 156, 158 (e.g., marker 154) is configured to provide an indication of whether sheath distal opening 104 is dorsal or ventral to fixation structure 136 to provide, for example, and indication of whether sheath 96 is positioned to permit deployment of fixation structure 136. In some examples, markers 152, 154, 156, 158 are configured to provide an indication to a clinician of when or whether fixation structure 136 has deployed within patient 14 as the clinician proximally withdraws sheath 96.

FIG. 13 illustrates an example sacral lead 28 including with lead body 62 including a helical member 160. Helical member 160 defines a helix configured to surround a longitudinal axis L defined by sacral lead 28. Helical member 160 is configured to flex and/or bend such that lead body 62 may define a curvature. Helical member 160 may be configured such that the curvature may vary depending on external forces placed on lead body 62. For example, helical member 160 may be configured to vary a defined curvature when force arise on lead body 62 due to motion of the patient once implanted, due to positioning by a clinician during implantation and/or removal, or other reasons. Further, helical member 160 may be configured to elongate to allow helical member 160 to flex and or bend. In examples, helical member 160 is configured to define a spacing H between a first helical turn 161 and a second helical turn 162. Helical member 160 may be configured such that the spacing H may vary (e.g., increase or decrease) when external forces are exerted on lead body 62 (e.g., due to movement of the patient, positioning by a clinician, or other reasons).

In examples, helical member 160 defines fixation structure 136. In examples, first helical turn 161 and second helical turn 162 are configured to engage tissue of patient 14 and resist translation of lead body 62 in the distal direction D and/or proximal direction P. For example, first helical turn 161 may be configured to engage the tissue such that, when lead body 62 experiences a force in the proximal direction P, a proximal edge of first helical turn 161 transmits some portion of the proximal force to the tissue and the tissue exerts an reaction force on the proximal edge opposite the proximal force, tending to reduce and/or substantially eliminate movement of lead body 62 which might result from the proximal force. First helical turn 161 may be configured such that, when lead body 62 experiences a force in the distal direction D, a distal edge of first helical turn 161 transmits some portion of the distal force to the tissue and the tissue exerts a reaction force on the distal edge opposite the distal force, tending to reduce and/or substantially eliminate movement of lead body 62. Second helical turn 162 and/or other helical turns defined by helical member 160 may be configured similarly.

In examples, a conductor (e.g., one or more of conductors 64 (FIG. 3)) defines helical member 160. The conductor may include an insulative coating substantially surrounding some portion of the conductor. In examples, the conductor defines one or more of electrodes 30, such as distal-most electrode 34. The defined electrode may be a portion of the conductor (e.g., a distal portion) where the insulative coating is removed.

Electrodes 34, 36, 38, 40 of leads 28 may be ring electrodes, segmented electrodes, partial ring electrodes or any suitable electrode configuration. Segmented and partial ring electrodes each extend along an arc less than 360 degrees (e.g., 90-120 degrees) around the outer perimeter of the respective lead 18, 20, 28. In some examples, segmented electrodes of lead 28 may be useful for targeting different fibers of the same or different nerves to generate different physiological effects (e.g., therapeutic effects). In examples, lead 28 is an axial lead. In some example, lead 28 may be, at least in part, paddle-shaped (e.g., a “paddle” lead), and may include an array of electrodes on a common surface, which may or may not be substantially flat. In some examples, one or more of electrodes 19, 20, 29 may be cuff electrodes that are configured to extend at least partially around a nerve (e.g., extend axially around an outer surface of a nerve). One or more of electrodes 30 may operate in a bipolar or multi-polar configuration with other electrodes, or may operate in a unipolar configuration referenced to a reference electrode (e.g., a reference electrode supported by medical device 16 or another device).

In general, medical device 16, external device 24, stimulation circuitry 44, and/or sensing circuitry 45 may comprise any suitable arrangement of hardware, alone or in combination with software and/or firmware, to perform the techniques of this disclosure. Medical device 16, external device 24, stimulation circuitry 44, and/or sensing circuitry 45 may include one or more processors, such as one or more microprocessors, DSPs, ASICs, FPGAs, or any other equivalent integrated or discrete logic circuitry, as well as any combinations of such components. Medical device 16, external device 24, stimulation circuitry 44, and/or sensing circuitry 45 may include a memory, such as RAM, ferroelectric RAM (FRAM), ROM, PROM, EPROM, EEPROM, flash memory, a hard disk, a CD-ROM, comprising executable instructions for causing the one or more processors to perform the actions attributed to them. Moreover, although medical device 16, external device 24, stimulation circuitry 44, and/or sensing circuitry 45 are described as separate circuitry, in some examples, medical device 16, external device 24, stimulation circuitry 44, and/or sensing circuitry 45 may be functionally integrated.

The techniques of this disclosure may be implemented in a wide variety of computing devices, medical devices, or any combination thereof. Any of the described units, circuitry or components may be implemented together or separately as discrete but interoperable logic devices. Depiction of different features as circuitry or units is intended to highlight different functional aspects and does not necessarily imply that such circuitry or units must be realized by separate hardware or software components. Rather, functionality associated with one or more circuitry or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various circuitry and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processor circuitry, as well as any combinations of such components, remote servers, remote client devices, or other devices. The term “processor circuitry” or “processor circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, circuitry or components may be implemented together or separately as discrete but interoperable logic devices. Depiction of different features as circuitry or units is intended to highlight different functional aspects and does not necessarily imply that such circuitry or units must be realized by separate hardware or software components. Rather, functionality associated with one or more circuitry or units may be performed by separate hardware or software components, or integrated within common or separate hardware or software components. For example, any circuitry described herein may include electrical circuitry configured to perform the features attributed to that particular circuitry, such as fixed function processor circuitry, programmable processor circuitry, or combinations thereof.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that may, over time, change (e.g., in RAM or cache).

The disclosure includes the following examples.

Example 1: A method of sensing and stimulation with a sacral lead, the method comprising: delivering a stimulation signal through one or more stimulation electrodes using one or more electrodes when the one or more electrodes are configured to operate as the one or more stimulation electrodes; and sensing, following delivery of the stimulation signal, an evoked signal with one or more sensing electrodes using the one or more electrodes when the one or more electrodes are configured to operate as the one or more sensing electrodes, wherein at least one of the stimulation electrodes is located within, dorsal, or ventral to a foramen of a sacrum of a patient, wherein at least one of the sensing electrodes is located within, dorsal, or ventral to the foramen of the sacrum of the patient, and wherein a lead body of the sacral lead supports at least some portion of the one or more electrodes, wherein the lead body is configured to position the at least some portion of the one or more electrodes within or ventral to the foramen.

Example 2: The method of example 1, wherein at least one of the stimulation electrodes is located ventral to a posterior opening of the foramen, and wherein the at least one of the sensing electrodes is located ventral to the posterior opening.

Example 3: The method of example 1 of example 2, wherein at least one of the stimulation electrodes is located ventral to an anterior opening of the foramen, and wherein at least one of the sensing electrodes is located ventral to a posterior opening of the foramen.

Example 4: The method of any of examples 1-3, wherein at least one of the stimulation electrodes is located within the foramen, and wherein at least one of the sensing electrodes is located ventral to an anterior opening of the foramen.

Example 5: The method of any of examples 1-4, wherein at least one of the stimulation electrodes is located ventral to a posterior opening of the foramen, and wherein at least one of the sensing electrodes is located dorsal to an anterior opening of the foramen.

Example 6: The method of any of examples 1-5, wherein at least of the one stimulation electrodes is located dorsal to an anterior opening of the foramen, and wherein at least one of the sensing electrodes is located ventral to a posterior opening of the foramen.

Example 7: The method of any of examples 1-6, wherein at least one of the stimulation electrodes is located dorsal to an anterior opening of the foramen, and wherein at least one of the sensing electrodes is located dorsal to the anterior opening of the foramen.

Example 8: The method of any of examples 1-7, wherein the at least one of the stimulation electrodes includes a first electrode, and wherein the at least one of the sensing electrodes includes the first electrode.

Example 9: The method of any of examples 1-8, wherein the one or more electrodes includes a plurality of electrodes, and wherein sensing the evoked signal with one or more sensing electrodes comprises sensing the evoked signal with the plurality of electrodes.

Example 10: The method of any of examples 1-9, wherein the evoked signal comprises a composite stimulation-evoked signal comprising a composite of signals generated by one or more signal sources of the patient, wherein a signal source comprises at least one of a muscle of the patient or a nerve of the patient.

Example 11: The method of any of examples 1-10, further comprising switching, using processing circuitry, the configuration of the first electrode at least from a first configuration to a second configuration, wherein the first electrode is configured to operate as one of the stimulation electrodes in the first configuration, and wherein the first electrode is configured to operate as one of the sensing electrodes in the second configuration.

Example 12: The method of any of examples 1-11, further comprising: extending the sacral lead, using the lead body, through a sheath lumen of an introducer sheath configured to extend within or ventral to the foramen, wherein the introducer sheath defines one or more windows defining one or more openings in a sheath wall of the introducer sheath, aligning, using the lead body, at least one window of the introducer sheath with at least one of the one or more stimulation electrodes or at least one of the one or more sensing electrodes.

Example 13: A method of sensing and stimulation with a sacral lead, the method comprising: extending, using a lead body of the sacral lead, the sacral lead through a sheath lumen of an introducer sheath configured to extend within or ventral to the foramen, wherein the introducer sheath defines one or more windows defining one or more openings in a sheath wall of the introducer sheath, wherein the lead body supports at least some portion of the one or more electrodes, and wherein the lead body is configured to position the at least some portion of the one or more electrodes within or ventral to the foramen; aligning, using the lead body, at least one window of the introducer sheath with at least one of the one or more electrodes; delivering a stimulation signal through one or more stimulation electrodes using the one or more electrodes when the one or more electrodes are configured to operate as the one or more stimulation electrodes; and sensing, following delivery of the stimulation signal, an evoked signal with one or more sensing electrodes using the one or more electrodes when the one or more electrodes are configured to operate as the one or more sensing electrodes, wherein the evoked signal includes a signal generated by a signal source of the patient in response to delivery of the stimulation signal, wherein the signal source includes at least one of a muscle of the patient or a nerve of the patient, wherein at least one of the stimulation electrodes is located within, dorsal, or ventral to a foramen of a sacrum of a patient, and wherein at least one of the sensing electrodes is located within, dorsal, or ventral to the foramen of the sacrum of the patient.

Example 14: A sacral lead system, comprising: one or more electrodes, wherein the one or more electrodes are configured to operate as one or more stimulation electrodes and one or more sensing electrodes; a sacral lead including a lead body, wherein the lead body supports at least some portion of the one or more electrodes, and wherein the lead body is configured to position at least some portion of the one or more electrodes within, dorsal to, or ventral to a foramen of a sacrum of a patient; and processing circuitry configured to: deliver, using the one or more electrodes configured to operate as the one or more stimulation electrodes, a stimulation signal; and sense, following delivery of the stimulation signal, and using the one or more electrodes configured to operate as the one or more sensing electrodes, an evoked signal.

Example 15: The system of example 14, wherein the sacral lead system is configured to at least one of: position at least one electrode configured to operate as the one or more stimulation electrodes ventral to a posterior opening of the foramen when at least one electrode configured to operate as the one or more sensing electrodes is positioned ventral to the posterior opening; position the at least one electrode configured to operate as the one or more stimulation electrodes ventral to an anterior opening of the foramen when the at least one electrode configured to operate as the one or more sensing electrodes is positioned ventral to the posterior opening; position the at least one electrode configured to operate as the one or more stimulation electrodes within the foramen when the at least one electrode configured to operate as the one or more sensing electrodes is positioned ventral to the anterior opening; position the at least one electrode configured to operate as the one or more stimulation electrodes ventral to the posterior opening when the at least one electrode configured to operate as the one or more sensing electrodes is positioned dorsal to the anterior opening; position the at least one electrode configured to operate as the one or more stimulation electrodes dorsal to the anterior opening when the at least one electrode configured to operate as the one or more sensing electrodes is positioned ventral to the posterior opening; or position the at least one electrode configured to operate as the one or more stimulation electrodes is dorsal to the anterior opening when the at least one electrode configured to operate as the one or more sensing electrodes is located dorsal to the anterior opening.

Example 16: The system of example 14 or example 15, wherein the lead body supports each of the one or more electrodes.

Example 17: The system of any of examples 14-16, wherein the one or more electrodes includes a first electrode, wherein the first electrode is configured to operate as one of the one or more stimulation electrodes in a first configuration and one of the one or more sensing electrodes in a second configuration, and wherein the processing circuitry is configured to switch the first electrode at least from the first configuration to the second configuration.

Example 18: The system of any of examples 14-17, wherein the processing circuitry is configured to sense a composite stimulation-evoked signal, wherein the composite stimulation-evoked signal includes a composite of signals generated by one or more signal sources of the patient, and wherein the evoked signal includes the composite stimulation-evoked signal.

Example 19: The system of any of examples 14-18, wherein the lead body includes one or more markers configured to be imaged by an imaging modality when the imaging modality images one or more anatomical features of the patient and at least one of the one or more markers.

Example 20: The system of any of examples 14-19, further comprising an introducer sheath defining a sheath lumen, wherein: the introducer sheath is configured to extend through the foramen; the lead body and the one or more electrodes are slidably translatable within the lumen; the introducer sheath includes one or more windows configured to at least one of: allow at least one stimulation electrode to emit the stimulation signal through the one or more windows when the lead body and the at least one stimulation electrode are positioned within the lumen and the at least one stimulation electrode is aligned with at least one of the one or more windows; or allow at least one sensing electrode to sense the evoked signal through the one or more windows when the lead body and the at least one sensing electrode are positioned within the lumen and the at least one sensing electrode is aligned with at least one of the one or more windows, wherein the introducer sheath is configured to align the at least one window and at least one of the at least one stimulation electrode or the at least one sensing electrode when the lead body slidably translates within the lumen.

Example 21: A method of sensing and stimulation with a sacral lead, the method comprising: generating a stimulation signal for delivery through one or more stimulation electrodes supported by a lead body of the sacral lead, wherein a stimulation electrode is located within or ventral to a foramen of a sacrum of a patient; and sensing an evoked signal with one or more sensing electrodes supported by the lead body of the sacral lead, wherein a sensing electrode is dorsal to an anterior opening of the foramen, and wherein the sensing electrode is intracorporeal to the patient when the stimulation electrode is located proximate a sacral nerve of the patient.

Example 22: The method of example 21, wherein the stimulation electrode is positioned proximate the anterior opening of the foramen.

Example 23: The method of example 21 or example 22, wherein the stimulation electrode is substantially ventral to the anterior opening.

Example 24: The method of any of examples 21-23, wherein the stimulation electrode is one of a plurality of electrodes supported by the lead body, and wherein the stimulation electrode is distal to every other electrode in the plurality.

Example 25: The method of any of examples 21-24, wherein the lead body supports a plurality of electrodes, wherein the stimulation electrode is a first electrode in the plurality, and further comprising generating the stimulation signal for delivery through the stimulation electrode and at least a second electrode in the plurality.

Example 26: The method of example 25, wherein the first electrode is substantially ventral to the anterior opening and the second electrode is substantially dorsal to the anterior opening.

Example 27: The method of any of examples 21-26, wherein the stimulation signal is a first stimulation signal, wherein the evoked signal sensed by the sensing electrode is a first evoked signal, and wherein the location of the stimulation electrode within or ventral to the foramen is a first location, the method further comprising: transmitting a signal indicative of the first evoked signal over a conductor within the lead body; generating a second stimulation signal for delivery through the stimulation electrode, wherein the stimulation electrode is located at a second location within or ventral to a foramen of a sacrum of a patient, and wherein the second location is anterior to the first location; and sensing a second evoked signal with the sensing electrode.

Example 28: The method of any of examples 21-27, further comprising: delivering the stimulation signal using the stimulation electrode; and sensing the evoked signal with the sensing electrode following delivery of the stimulation signal.

Example 29: The method of any of examples 21-28, wherein the sensing electrode is located within the foramen.

Example 30: The method of any of examples 21-29, wherein the sensing electrode is located dorsal to a posterior opening of the sacrum.

Example 31: The method of any of examples 21-30, wherein the lead body supports a plurality of electrodes and wherein the stimulation electrode is one of the plurality of electrodes, and wherein the lead body is configured to position the plurality of electrodes within the foramen.

Example 32: The method of example 31, wherein the sensing electrode is another of the plurality of electrodes.

Example 33: The method of any of examples 21-32, wherein the lead body includes an imaging marker configured to define a displacement between the imaging marker and the stimulation electrode on the lead body.

Example 34: The method of any of examples 21-33, wherein the sensing electrode is supported by the lead body at first location on the lead body and the stimulation electrode is supported by the lead body at second location on the lead body, wherein the first location is proximal to the second location.

Example 35: The method of any of examples 21-34, wherein the sacral lead comprises: a first conductor electrically connected to the sensing electrode; and a second conductor electrically connected to the stimulation electrode, wherein the first conductor is electrically isolated from the second conductor.

Example 36: The method of any of examples 21-35, further comprising an introducer sheath defining a sheath lumen, wherein: the introducer sheath is configured to extend through the foramen, the lead body at and least the stimulation electrode is slidably translatable within the lumen; the introducer sheath includes a window configured to allow the stimulation electrode to emit the stimulation signal from within the lumen to an exterior of the introducer sheath when the stimulation electrode is aligned with the window; and the introducer sheath is configured to align the window and the stimulation electrode when the lead body slidably translates within the lumen.

Example 37: The method of example 36, wherein: the stimulation electrode is a first electrode and further comprising a second electrode supported by the lead body; the window is configured to allow both the first electrode and the second electrode to emit the stimulation signal from the position within the lumen to the exterior of the introducer sheath when the window aligns with the first electrode and the second electrode; and the introducer sheath is configured to align the window with the first electrode and the second electrode when the lead body slidably translates within the lumen.

Example 38: The method of example 36, wherein: the lead body supports a plurality of electrodes; each electrode is configured to deliver the stimulation signal; the introducer sheath includes a plurality of windows; each individual window is configured to allow an individual electrode to emit the stimulation signal from within the lumen to the exterior of the introducer sheath when the individual electrode is aligned with the individual window; and the introducer sheath is configured to vary the number of individual electrodes aligned with individual windows when the lead body slidably translates within the lumen.

Example 39: The method of any of examples 21-38, further comprising a plurality of conductors, wherein the lead body supports a plurality of electrodes including the stimulation electrode and the sensing electrode, wherein each individual conductor in the plurality of conductors is electrically connected to one individual electrode, and wherein the each individual conductor is electrically isolated from every other conductor in the plurality of conductors.

Example 40: The method of any of examples 21-39, wherein the sensing electrode defines a first effective surface area and the stimulation electrode defines a second effective surface area, wherein the second effective surface area is greater than the first effective surface area.

Example 41: The method of any of examples 21-40, wherein the sensing electrode defines a first effective surface area defining a first input impedance and the stimulation electrode defines a second effective surface area defining a second input impedance, wherein the first input impedance is greater than the second input impedance when the stimulation electrode is positioned in proximate the sacral nerve and the sensing electrode is positioned proximal to the foramen and intracorporeal to the patient.

Example 42: The method of any of examples 21-41, wherein the lead body includes a fixation structure configured to resist a translation of the stimulation electrode when the stimulation electrode is positioned proximate the sacral nerve.

Example 43: The method of any of examples 21-42, wherein the lead body defines a first spacing between the sensing electrode and the stimulation electrode, and wherein the lead body is configured to extend to define a second spacing between the sensing electrode and the stimulation electrode, wherein the second spacing is greater than the first spacing.

Example 44: The method of example 43, wherein the lead body includes a helical coil defining the first spacing, wherein the helical coil is configured to extend to define the second spacing.

Example 45: The method of example 44, wherein the helical coil is configured to resist a translation of the stimulation electrode when the stimulation electrode is positioned distal to the foramen.

Example 46: The method of any of examples 21-45, wherein: the lead body includes a connector in electrical communication with the stimulation electrode and the sensing electrode, the connector is configured to mechanically mate with a terminal of an external device, the connector is configured to electrically connect to the terminal when the connector mechanically mates with the terminal, and the sensing electrode is distal to the first terminal.

Example 47: The method of example 46 wherein the connector is configured to electrically connect the sensing electrode and the terminal and electrically connect the stimulation electrode and the terminal when connector mechanically mates with the first terminal.

Example 48: The method of example 46 or 47, wherein the stimulation electrode is configured to receive the generated stimulation signal from the connector and transmit the stimulation signal to the patient, and wherein the sensing electrode is configured to receive the evoked signal from the patient and deliver a signal indicative of the evoked signal to the connector.

Example 49: The method of any of examples 21-48, wherein the evoked signal comprises a composite stimulation-evoked signal comprising a composite of signals generated by two or more signal sources in response to the stimulation signal.

Example 50: The method of example 49, wherein the two or more signal sources comprises one or more of: two or more muscles of the patient; two or more nerves of the patient; or at least one muscle and at least one nerve of the patient.

Example 51: A sacral lead system, comprising: stimulation circuitry configured to generate a stimulation signal for delivery to a patient; sensing circuitry configured to receive a signal indicative of an evoked signal produced by the patient; and a sacral lead including a lead body supporting one or more electrodes, wherein one or more electrodes are a stimulation electrode operably connected to the stimulation circuitry, wherein one or more electrodes are a sensing electrode operably connected to the sensing circuitry, wherein the sacral lead is configured to deliver the stimulation signal using a stimulation electrode and sense the evoked signal using a sensing electrode, and wherein the sacral lead is configured to position the stimulation electrode within or ventral to a foramen of a sacrum of a patient when the sensing electrode is dorsal to an anterior opening of the foramen and intracorporeal to the patient.

Example 52: The sacral lead system of example 51, wherein the sacral lead is configured to position the stimulation electrode proximate the anterior opening of the foramen when the sensing electrode is dorsal to an anterior opening of the foramen and intracorporeal to the patient.

Example 53: The sacral lead system of example 51 or example 52, wherein the sacral lead is configured to position the stimulation electrode substantially ventral to the anterior opening when the sensing electrode is dorsal to an anterior opening of the foramen and intracorporeal to the patient.

Example 54: The sacral lead system of any of examples 51-53, wherein the stimulation electrode is one of a plurality of electrodes supported by the lead body, and wherein the stimulation electrode is distal to every other electrode in the plurality.

Example 55: The sacral lead system of any of examples 51-54, wherein the lead body supports a plurality of electrodes, wherein the stimulation electrode is a first electrode in the plurality, and further comprising generating the stimulation signal for delivery through the stimulation electrode and at least a second electrode in the plurality.

Example 56: The sacral lead system of example 51-55, wherein the sacral lead is configured to position the first electrode substantially ventral to the anterior opening when the second electrode is substantially dorsal to the anterior opening.

Example 57: The sacral lead system of any of examples 51-56, wherein the sacral lead system is configured such that the stimulation circuitry delivers the stimulation signal using the stimulation electrode and the sensing circuitry receives the signal indicative of the evoked signal following delivery of the stimulation signal.

Example 58: The sacral lead system of any of examples 51-57, wherein the sacral lead is configured to position the sensing electrode within the foramen of the patient when the stimulation electrode is positioned within or ventral to the foramen of the patient.

Example 59: The sacral lead system of any of examples 51-58, wherein the sacral lead is configured to position the sensing electrode dorsal to a posterior opening of the sacrum when the stimulation electrode is positioned within or ventral to the foramen of the patient.

Example 60: The sacral lead system of any of examples 51-59, wherein the lead body supports a plurality of electrodes and wherein the stimulation electrode is one of the plurality of electrodes, and wherein the lead body is configured to position the plurality of electrodes within the foramen.

Example 61: The sacral lead system of example 60, wherein the sensing electrode is another of the plurality of electrodes.

Example 62: The sacral lead system of any of examples 51-61, wherein the lead body includes an imaging marker configured to define a displacement between the imaging marker and the stimulation electrode on the lead body.

Example 63: The sacral lead system of any of examples 51-62, wherein the sensing electrode is supported by the lead body at first location on the lead body and the stimulation electrode is supported by the lead body at second location on the lead body, wherein the first location is proximal to the second location.

Example 64: The sacral lead system of any of examples 51-63, wherein the sacral lead comprises: a first conductor electrically connected to the sensing electrode; and a second conductor electrically connected to the stimulation electrode, wherein the first conductor is electrically isolated from the second conductor.

Example 65: The sacral lead system of any of examples 51-64, further comprising an introducer sheath defining a sheath lumen, wherein: the introducer sheath is configured to extend through the foramen, the lead body at and least the stimulation electrode is slidably translatable within the lumen; the introducer sheath includes a window configured to allow the stimulation electrode to emit the stimulation signal from within the lumen to an exterior of the introducer sheath when the stimulation electrode is aligned with the window; and the introducer sheath is configured to align the window and the stimulation electrode when the lead body slidably translates within the lumen.

Example 66: The sacral lead system of example 65, wherein: the stimulation electrode is a first electrode and further comprising a second electrode supported by the lead body; the window is configured to allow both the first electrode and the second electrode to emit the stimulation signal from the position within the lumen to the exterior of the introducer sheath when the window aligns with the first electrode and the second electrode; and the introducer sheath is configured to align the window with the first electrode and the second electrode when the lead body slidably translates within the lumen.

Example 67: The sacral lead system of example 66, wherein: the lead body supports a plurality of electrodes; each electrode is configured to deliver the stimulation signal; the introducer sheath includes a plurality of windows; and each individual window is configured to allow an individual electrode to emit the stimulation signal from within the lumen to the exterior of the introducer sheath when the individual electrode is aligned with the individual window; and the introducer sheath is configured to vary the number of individual electrodes aligned with individual windows when the lead body slidably translates within the lumen.

Example 68: The sacral lead system of any of examples 51-67, further comprising a plurality of conductors, wherein the lead body supports a plurality of electrodes including the stimulation electrode and the sensing electrode, wherein each individual conductor in the plurality of conductors is electrically connected to one individual electrode, and wherein the each individual conductor is electrically isolated from every other conductor in the plurality of conductors.

Example 69: The sacral lead system of any of examples 51-68, wherein the sensing electrode defines a first effective surface area and the stimulation electrode defines a second effective surface area, wherein the second effective surface area is greater than the first effective surface area.

Example 70: The sacral lead system of any of examples 51-69, wherein the sensing electrode defines a first effective surface area defining a first input impedance and the stimulation electrode defines a second effective surface area defining a second input impedance, wherein the first input impedance is greater than the second input impedance when the stimulation electrode is positioned proximate the sacral nerve and the sensing electrode is positioned proximal to the foramen and intracorporeal to the patient.

Example 71: The sacral lead system of any of examples 51-70, wherein the lead body includes a fixation structure configured to resist a translation of the stimulation electrode when the stimulation electrode is positioned proximate the sacral nerve.

Example 72: The sacral lead system of any of examples 51-71, wherein the lead body defines a first spacing between the sensing electrode and the stimulation electrode, and wherein the lead body is configured to extend to define a second spacing between the sensing electrode and the stimulation electrode, wherein the second spacing is greater than the first spacing.

Example 73: The sacral lead system of example 72, wherein the lead body includes a helical coil defining the first spacing, wherein the helical coil is configured to extend to define the second spacing.

Example 74: The sacral lead system of example 73, wherein the helical coil is configured to resist a translation of the stimulation electrode when the stimulation electrode is positioned distal to the foramen.

Example 75: The sacral lead system of any of examples 51-74, wherein: the lead body includes a connector in electrical communication with the stimulation electrode and the sensing electrode, the connector is configured to mechanically mate with a terminal of an external device, the connector is configured to electrically connect to the terminal when the connector mechanically mates with the terminal, and the sensing electrode is distal to the first terminal.

Example 76: The sacral lead system of example 75, wherein the connector is configured to electrically connect the sensing electrode and the terminal and electrically connect the stimulation electrode and the terminal when connector mechanically mates with the first terminal.

Example 77: The sacral lead system of examples 75 or example 77, wherein the stimulation electrode is configured to receive the generated stimulation signal from the connector and transmit the stimulation signal to the patient, and wherein the sensing electrode is configured to receive the evoked signal from the patient and deliver a signal indicative of the evoked signal to the connector.

Example 78: The sacral lead system of any of examples 51-77, wherein the evoked signal comprises a composite stimulation-evoked signal comprising a composite of signals generated by two or more signal sources in response to the stimulation signal.

Example 79: The sacral lead system of any of examples 51-78, wherein the two or more signal sources comprises one or more of: two or more muscles of the patient; two or more nerves of the patient; or at least one muscle and at least one nerve of the patient.

Various examples have been described herein. Any combination of the described operations or functions is contemplated.

We claim:

1. A method of sensing and stimulation with a sacral lead, the method comprising:
delivering a stimulation signal through one or more stimulation electrodes using one or more electrodes when the one or more electrodes are configured to operate as the one or more stimulation electrodes; and
sensing, following delivery of the stimulation signal, an evoked signal with one or more sensing electrodes using the one or more electrodes when the one or more electrodes are configured to operate as the one or more sensing electrodes,
wherein at least one of the stimulation electrodes is located within, dorsal, or ventral to a foramen of a sacrum of a patient,
wherein at least one of the sensing electrodes is located within, dorsal, or ventral to the foramen of the sacrum of the patient, and
wherein a lead body of the sacral lead supports at least some portion of the one or more electrodes, wherein the lead body is configured to position the at least some portion of the one or more electrodes within or ventral to the foramen.

2. The method of claim 1, wherein the at least one of the stimulation electrodes is located ventral to a posterior opening of the foramen, and wherein the at least one of the sensing electrodes is located ventral to the posterior opening.

3. The method of claim 1, wherein the at least one of the stimulation electrodes is located ventral to an anterior opening of the foramen, and wherein the at least one of the sensing electrodes is located ventral to a posterior opening of the foramen.

4. The method of claim 1, wherein the at least one of the stimulation electrodes is located within the foramen, and wherein the at least one of the sensing electrodes is located ventral to an anterior opening of the foramen.

5. The method of claim 1, wherein the at least one of the stimulation electrodes is located ventral to a posterior opening of the foramen, and wherein the at least one of the sensing electrodes is located dorsal to an anterior opening of the foramen.

6. The method of claim 1, wherein the at least one at least one of the stimulation electrodes is located dorsal to an anterior opening of the foramen, and wherein the at least one of the sensing electrodes is located ventral to a posterior opening of the foramen.

7. The method of claim 1, wherein the at least one at least one of the stimulation electrodes is located dorsal to an anterior opening of the foramen, and wherein the at least one of the sensing electrodes is located dorsal to the anterior opening of the foramen.

8. The method of claim 1, wherein the at least one of the stimulation electrodes includes a first electrode, and wherein the at least one of the sensing electrodes includes the first electrode.

9. The method of claim 1, wherein the one or more electrodes includes a plurality of electrodes, and wherein sensing the evoked signal with one or more sensing electrodes comprises sensing the evoked signal with the plurality of electrodes.

10. The method of claim 1, wherein the evoked signal comprises a composite stimulation-evoked signal comprising a composite of signals generated by one or more signal sources of the patient, wherein a signal source comprises at least one of a muscle of the patient or a nerve of the patient.

11. The method of claim 1, further comprising switching, using processing circuitry, the configuration of the first electrode at least from a first configuration to a second configuration,
wherein the first electrode is configured to operate as one of the stimulation electrodes in the first configuration, and
wherein the first electrode is configured to operate as one of the sensing electrodes in the second configuration.

12. The method of claim 1, further comprising:
extending the sacral lead, using the lead body, through a sheath lumen of an introducer sheath configured to extend within or ventral to the foramen, wherein the introducer sheath defines one or more windows defining one or more openings in a sheath wall of the introducer sheath,
aligning, using the lead body, at least one window of the introducer sheath with at least one of the one or more stimulation electrodes or at least one of the one or more sensing electrodes.

13. A method of sensing and stimulation with a sacral lead, the method comprising:
extending, using a lead body of the sacral lead, the sacral lead through a sheath lumen of an introducer sheath configured to extend dorsal to or within the foramen, wherein the introducer sheath defines one or more windows defining one or more openings in a sheath wall of the introducer sheath, wherein the lead body supports at least some portion of the one or more electrodes, and wherein the lead body is configured to position the at least some portion of the one or more electrodes dorsal to, within or ventral to the foramen;
aligning, using the lead body, at least one window of the introducer sheath with at least one of the one or more electrodes;
delivering a stimulation signal through one or more stimulation electrodes using the one or more electrodes when the one or more electrodes are configured to operate as the one or more stimulation electrodes; and
sensing, following delivery of the stimulation signal, an evoked signal with one or more sensing electrodes using the one or more electrodes when the one or more electrodes are configured to operate as the one or more sensing electrodes,
wherein the evoked signal includes a signal generated by a signal source of the patient in response to delivery of the stimulation signal, wherein the signal source includes at least one of a muscle of the patient or a nerve of the patient,
wherein at least one of the stimulation electrodes is located within, dorsal, or ventral to a foramen of a sacrum of a patient, and
wherein at least one of the sensing electrodes is located within, dorsal, or ventral to the foramen of the sacrum of the patient.

14. A sacral lead system, comprising:
one or more electrodes, wherein the one or more electrodes are configured to operate as one or more stimulation electrodes and one or more sensing electrodes;
a sacral lead including a lead body, wherein the lead body supports at least some portion of the one or more electrodes, and wherein the lead body is configured to position at least some portion of the one or more electrodes within, dorsal to, or ventral to a foramen of a sacrum of a patient; and
processing circuitry configured to:
deliver, using the one or more electrodes configured to operate as the one or more stimulation electrodes, a stimulation signal; and
sense, following delivery of the stimulation signal, and using the one or more electrodes configured to operate as the one or more sensing electrodes, an evoked signal.

15. The system of claim 14, wherein the sacral lead system is configured to at least one of:
position at least one electrode configured to operate as the one or more stimulation electrodes ventral to a posterior opening of the foramen when at least one electrode configured to operate as the one or more sensing electrodes is positioned ventral to the posterior opening;
position the at least one electrode configured to operate as the one or more stimulation electrodes ventral to an anterior opening of the foramen when the at least one electrode configured to operate as the one or more sensing electrodes is positioned ventral to the posterior opening;
position the at least one electrode configured to operate as the one or more stimulation electrodes within the foramen when the at least one electrode configured to operate as the one or more sensing electrodes is positioned ventral to the anterior opening;
position the at least one electrode configured to operate as the one or more stimulation electrodes ventral to the posterior opening when the at least one electrode configured to operate as the one or more sensing electrodes is positioned dorsal to the anterior opening;
position the at least one electrode configured to operate as the one or more stimulation electrodes dorsal to the anterior opening when the at least one electrode configured to operate as the one or more sensing electrodes is positioned ventral to the posterior opening; or
position the at least one electrode configured to operate as the one or more stimulation electrodes is dorsal to the anterior opening when the at least one electrode configured to operate as the one or more sensing electrodes is located dorsal to the anterior opening.

16. The system of claim 14, wherein the lead body supports each of the one or more electrodes.

17. The system of claim 14,
wherein the one or more electrodes includes a first electrode,
wherein the first electrode is configured to operate as one of the one or more stimulation electrodes in a first configuration and one of the one or more sensing electrodes in a second configuration, and
wherein the processing circuitry is configured to switch the first electrode at least from the first configuration to the second configuration.

18. The system of claim 14, wherein the processing circuitry is configured to sense a composite stimulation-evoked signal, wherein the composite stimulation-evoked signal includes a composite of signals generated by one or more signal sources of the patient, wherein a signal source of the patient comprises at least one of a muscle of the patient or a nerve of the patient, and wherein the evoked signal includes the composite stimulation-evoked signal.

19. The system of claim 14, wherein the lead body includes one or more markers configured to be imaged by an imaging modality when the imaging modality images one or more anatomical features of the patient and at least one of the one or more markers.

20. The system of claim 14, further comprising an introducer sheath defining a sheath lumen, wherein:
the introducer sheath is configured to extend through the foramen;
the lead body and the one or more electrodes are slidably translatable within the lumen;
the introducer sheath includes one or more windows configured to at least one of:
allow at least one stimulation electrode to emit the stimulation signal through the one or more windows when the lead body and the at least one stimulation electrode are positioned within the lumen and the at least one stimulation electrode is aligned with at least one of the one or more windows; or
allow at least one sensing electrode to sense the evoked signal through the one or more windows when the lead body and the at least one sensing electrode are positioned within the lumen and the at least one sensing electrode is aligned with at least one of the one or more windows,
wherein the introducer sheath is configured to align the at least one window and at least one of the at least one stimulation electrode or the at least one sensing electrode when the lead body slidably translates within the lumen.
